(19) United States
(12) Patent Application Publication
(10) Pub. No.: US 2024/0189774 A1
(43) Pub. Date: Jun. 13, 2024

(54) REAL-TIME AUTOMATED MONITORING AND CONTROL OF ULTRAFILTRATION/DIAFILTRATION (UF/DF) CONDITIONING AND DILUTION PROCESSES

(71) Applicant: Genentech, Inc., South San Francisco, CA (US)

(72) Inventors: Nima SAMMAKNEJAD, San Mateo, CA (US); Victor M. SAUCEDO, Sunnyvale, CA (US); John R. LAZZARESCHI, San Mateo, CA (US); Nicholas T. WOON, San Francisco, CA (US); Govindaraj SRIDHARAN, Santa Clara, CA (US); Bibi EPHRAIM, Oakland, CA (US)

(21) Appl. No.: 18/536,490

(22) Filed: Dec. 12, 2023

Related U.S. Application Data
(60) Provisional application No. 63/432,033, filed on Dec. 12, 2022.

(51) Int. Cl. B01D 61/22; B01D 61/02; B01D 61/12; B01D 61/14

(52) U.S. Cl. CPC B01D 61/22; B01D 61/027; B01D 61/12; B01D 61/145; B01D 2313/702

(57) ABSTRACT

Methods and system for real-time monitoring and control of an ultrafiltration/diafiltration (UF/DF) conditioned pool in a UF/DF recovery tank are disclosed. In various embodiments, a UF/DF pool is received at a recovery tank for conditioning or dilution by a buffer. Inline Raman measurements of the conditioned UF/DF pool may be performed and provided to a trained machine learning model as input. The machine learning model can then predict product quality attributes of the UF/DF conditioned pool, examples of said product quality attributes including protein concentration and osmolality of the conditioned UF/DF pool.

### CROSS-REFERENCE TO RELATED APPLICATION

This application claims priority to U.S. provisional patent application Ser. No. 63/432,033 filed Dec. 12, 2023, the entire content of which is incorporated herein by reference and relied upon.

### FIELD

This description is generally directed towards real-time (e.g., online, in-line, etc.) automated monitoring and control of an ultrafiltration/diafiltration (UF/DF) conditioning or dilution processes. More specifically, this description provides methods and systems for real-time automated monitoring and controlling of product quality attributes of a UF/DF conditioning or dilution process downstream from a UF/DF process with the use of machine learning models. As the outcome of such methods and systems, predictions and/or measurements of the quality attributes at the end of (target) UF/DF conditioning can be used for instant release of the drug substance, replacing manual quality control tests.

### BACKGROUND

Large scale manufacturing of biomolecules can be highly involved processes and usually include robust quality control mechanisms such as the sampling of the biomolecules or cell culture media for offline measurements of various product quality attributes of the manufacturing processes. For example, monoclonal antibodies purified from cell culture media or product may be received in a UF/DF recovery vessel for conditioning, and samples of the monoclonal antibodies may be taken from the recovery vessel for offline evaluation of product quality attributes. But such quality control mechanisms can be time- and resource-intensive. Accordingly, there is a need for techniques that facilitate rapid and inline monitoring and control of product quality attributes of UF/DF conditioned pools in recovery vessels.

### SUMMARY

The present disclosure provides techniques that allow product quality to be automatically monitored and controlled in real-time, avoiding undesired process deviations Further, final measurements of the quality attributes at the end of UF/DF conditioning can be used to facilitate instant release of the product. In various embodiments, according to a first aspect, there is provided a method of predicting one or more product quality attributes of a conditioned ultrafiltration/diafiltration (UF/DF) pool in a bioprocess. In various embodiments, the method comprises receiving, from a Raman spectrometer operationally coupled to a recovery vessel that contains a conditioned UF/DF pool, a Raman measurement of the conditioned UF/DF pool, wherein the recovery vessel is downstream from UF/DF operation and receives a UF/DF pool for conditioning with a buffer to form the conditioned UF/DF pool. The Raman measurement may be received as an inline measurement. For example, the Raman measurement may be received in real time or near real time. The Raman measurement may be a measurement of the conditioned UF/DF pool at a current time point, and predicting the one or more product quality attributes of the conditioned UF/DF pool may comprise predicting the one or more product quality attributes of the conditioned UF/DF pool at the current time point. Further, the method comprises predicting the one or more product quality attributes of the conditioned UF/DF pool using a machine learning model that has been trained to receive, as input, a Raman measurement of a conditioned UF/DF pool and generate, as output, one or more predicted product quality attributes of the conditioned UF/DF pool. The machine learning model may have been trained using training data comprising a plurality of Raman measurements of a conditioned UF/DF pool and corresponding measurements of the one or more product quality attributes of the conditioned UF/DF pool.

In embodiments, the one or more product quality attributes include osmolality of the conditioned UF/DF pool.

In embodiments, a product of the bioprocess comprises one or more proteins in a solution, wherein the one or more product quality attributes include protein concentration of the conditioned UF/DF pool.

In embodiments, the UF/DF pool is a purified UF/DF pool received at the recovery vessel from an upstream ultrafiltration and diafiltration process

In embodiments, the conditioned UF/DF pool includes an antibody.

In embodiments, the antibody is a monoclonal antibody (mAb).

In embodiments, the machine learning model has been trained to predict the one or more product quality attributes using a training dataset that includes the one or more product quality attributes of conditioned UF/DF pools conditioned with differing amounts of the buffer added therein and associated Raman measurements of the conditioned UF/DF pools conditioned with the differing amounts of the buffer.

In embodiments, the trained machine learning model is a trained multivariate linear model, a trained neural network, a trained deep learning model, or a trained ensemble model.

In embodiments, the trained machine learning model is one of a design of experiments (DOE) partial least squares (PLS) regression model, a trend analytics (TA) partial least squares (PLS) regression model, or a combination thereof.

In embodiments, the method further comprises generating an indication indicating whether the conditioned UF/DF pool is ready for instant release based on the one or more predicted product quality attributes. The indication may be generated by comparing the one or more predicted product quality attributes to one or more predetermined thresholds for the respective one or more predicted product quality attributes. In embodiments, predicting the one or more product quality attributes of the conditioned UF/DF pool comprises predicting the one or more product quality attributes of the conditioned UF/DF pool at a current timepoint. In other embodiments, predicting the one or more product quality attributes of the conditioned UF/DF pool comprises predicting the one or more product quality attributes of the conditioned UF/DF pool at a future timepoint (e.g., an end point of the conditioning process).

In embodiments, the steps of receiving the Raman measurement and predicting the one or more product quality attributes is repeated one or more times at a predetermined frequency.

In embodiments, receiving, as input, a Raman measurement of a conditioned UF/DF pool comprises receiving, as input, one or more of: an image data of a spectra corresponding to the Raman measurement, a series of peaks in the spectra, a data series comprising an intensity value for each of a plurality of wavenumbers, and a stride between a minimum and a maximum of the plurality of wavenumbers in the spectra, or a combination thereof.

In various embodiments, according to a second aspect, there is provided a method of controlling a UF/DF pool conditioning process. In various embodiments, the method comprises receiving, at a processor and from a Raman spectrometer operationally coupled to a recovery vessel that contains a conditioned UF/DF pool, a first Raman measurement of the conditioned UF/DF pool, wherein the recovery vessel is downstream from an UF/DF operation, and receives a UF/DF pool for conditioning with a buffer to form the conditioned UF/DF pool. Further, the method comprises predicting, by the processor, using a trained machine learning model receiving the first Raman measurement as an input, one or more product quality attributes of the conditioned UF/DF pool, wherein the one or more product quality attributes include a first protein concentration of the conditioned UF/DF pool. In addition, the method comprises computing, by the processor, a weight indicator of the recovery vessel that corresponds to a second protein concentration of the conditioned UF/DF pool that is different from the first protein concentration of the conditioned UF/DF pool. The method further comprises outputting, by the processor, an indication of whether to add or cease adding the buffer into the recovery vessel based on the computed weight indicator of the recovery vessel.

In embodiments, outputting, by the processor, an indication of whether to add or cease adding the buffer into the recovery vessel comprises the processor transmitting a buffer pump signal to a buffer pump operationally coupled to the recovery vessel, the buffer pump signal configured to instruct the buffer pump to add or cease adding the buffer into the recovery vessel.

In embodiments, the method comprises computing, by the processor, a weight indicator of the recovery vessel that corresponds to the first protein concentration of the conditioned UF/DF, and determining, by the processor, an amount of buffer to add into the recovery vessel based on the weight indicator of the recovery vessel that corresponds to the first protein concentration and the weight indicator of the recovery vessel that corresponds to the second protein concentration. For example, the amount of buffer may depend on the difference between the weight indicator of the recovery vessel that corresponds to the first protein concentration and the weight indicator of the recovery vessel that corresponds to the second protein concentration.

In embodiments, the method further comprises determining, by the processor, a flow rate for the determined amount of buffer to add into the recovery vessel based on the weight indicator of the recovery vessel that corresponds to the first protein concentration and the weight indicator of the recovery vessel that corresponds to the second protein concentration.

In embodiments, determining the flow rate for the determined amount of buffer to add into the recovery vessel may comprises determining whether to increase or decrease the flow rate.

In embodiments, the indication of whether to add or cease adding the buffer comprises an indication of the determined amount of buffer to add into the recovery vessel. The second protein concentration may be a predetermined concentration, such as, for example, a target protein concentration for the product. The step of computing, by the processor, a weight indicator of the recovery vessel that corresponds to a protein concentration of the conditioned UF/DF pool may use a predetermined relationship between the weight indicator and the protein concentration. The predetermined relationship may be in the form of a mathematical function or a lookup table. The weight indicator may be a value that is indicative of the amount (e.g., volume) of product in the recovery vessel.

In embodiments, the indication of whether to add or cease adding the buffer comprises an indication of a flow rate for the determined amount of buffer to add into the recovery vessel.

In embodiments, the first Raman measurement corresponds to a current time point of the conditioned UF/DF pool. In some aspects, the predicted first protein concentration and/or weight indicator may correspond to the current time point of the conditioned UF/DF pool.

In embodiments, the processor may be in wired or wireless communication with the Raman spectrometer. In some instances, the processor may be in remote communication with the Raman spectrometer. In some aspects, a latency between the receiving and the outputting is no greater than about 2 s.

In embodiments, the processor is an edge node, a processor that executes a virtual machine, a processor of a cloud server, or a processor of a cloud serverless solution.

In embodiments, the one or more product quality attributes of the conditioned UF/UD pool further include an osmolality of the conditioned UF/DF pool, wherein the predicting includes predicting an osmolality of the conditioned UF/DF pool based on the analysis of the first Raman measurement.

In embodiments, the trained machine learning model may be configured to receive, as input, the first Raman measurement, and generate, as outputs, the first protein concentration of the conditioned UF/DF pool and the osmolality of the conditioned UF/DF pool. The trained machine learning model may comprise a plurality of models, including a first trained machine learning model configured to take as input the first Raman measurement and produce as output the first protein concentration of the conditioned UF/DF pool, and a second trained machine learning model configured to take as input the first Raman measurement and produce as output the osmolality of the conditioned UF/DF pool. The trained machine learning model may include one or more models each configured to take, as input, the first Raman measurement, and generate, as outputs, both the first protein concentration of the conditioned UF/DF pool and the osmolality of the conditioned UF/DF pool.

In embodiments, the method further comprises: receiving, at the processor and from the Raman spectrometer, a second Raman measurement of the conditioned UF/DF pool after the transmitting. The method may further comprise: predicting, using the processor using the trained machine learning model taking as input the second Raman measurement, a third protein concentration of the conditioned UF/DF pool. Additionally, the method may further comprise: comparing, by the processor, the second protein concentration to the third protein concentration to determine effectiveness of the addition of the buffer into the recovery vessel.

In embodiments, the method does not use any measurement obtained by extracting a sample of the conditioned UF/DF pool from the recovery vessel.

In embodiments, the conditioned UF/DF pool includes an antibody. For example, the antibody may comprise a monoclonal antibody (mAb).

In embodiments, the method may further comprise: responsive to an indication to add the buffer, adding the buffer in an amount sufficient to increase a weight indicator of the recovery vessel to within a weight threshold of the computed weight indicator.

In embodiments, the trained machine learning model is a trained multivariate linear model, a trained neural network, a trained deep learning model, or a trained ensemble model.

In embodiments, the trained machine learning model is one of a design of experiments (DOE) partial least squares (PLS) regression model, a trend analytics (TA) partial least squares (PLS) regression model, or a combination thereof.

In embodiments, the method may further comprise: receiving, at the processor and from the Raman spectrometer, a second Raman measurement of the conditioned UF/DF pool after the outputting. The method may further comprise: predicting, by the processor using the trained machine learning model taking as input the second Raman measurement, a third protein concentration of the conditioned UF/DF pool. The method may further comprise: generating, by the processor, an indication indicating whether the conditioned UF/DF pool is ready for instant release based on the predicted third protein concentration.

In various embodiments, according to a third aspect, there is provided a system comprising an UF/DF pool recovery system and a communication module. In various embodiments, the UF/DF pool recovery system is downstream from an UF/DF operation and is configured to receive and store a UF/DF pool conditioned with a buffer. Further, the UF/DF pool recovery system includes a Raman spectrometer operationally coupled to the recovery vessel and configured to perform a Raman measurement of the conditioned UF/DF pool. In various embodiments, the communication module is operationally coupled to a remote computing platform and the Raman spectrometer and configured to: (i) receive the Raman measurement from the Raman spectrometer and upload the Raman measurement to the remote computing platform for prediction of one or more product quality attributes by the remote computing platform based on the Raman measurement; and (ii) transmit a signal, received from the remote computing platform and related to the one or more product quality attributes, to the UF/DF pool recovery system. In various embodiments, a latency between the receiving of the Raman measurement at the communication module and the transmission of the signal to the UF/DF pool recovery system satisfies a latency threshold.

In embodiments, the system may further comprise the remote computing platform. The remote computing platform comprises a processor configured to receive the Raman measurement from the communication module, predict one or more product quality attributes, and output a signal related to the one or more product quality attributes.

In embodiments, the one or more product quality attributes include a first protein concentration of the conditioned UF/DF pool. The processor is further configured to compute a weight indicator of the recovery vessel that corresponds to a second protein concentration of the conditioned UF/DF pool that is different from the first protein concentration of the conditioned UF/DF pool. The UF/DF pool recovery system may further comprise a buffer pump operationally coupled to the recovery vessel. The signal may be a buffer pump signal configured to instruct the buffer pump to add or cease adding the buffer into the recovery vessel based on a weight indicator of the recovery vessel.

In embodiments, the one or more product quality attributes may include an osmolality of the conditioned UF/DF pool.

In embodiments, the latency threshold may be about 2 seconds (e.g., 2 seconds).

In embodiments, the conditioned UF/DF pool includes an antibody. For example, the antibody may comprise a monoclonal antibody (mAb).

In various embodiments, according to a fourth aspect, there is provided a computer-implemented method for providing a tool for predicting a product quality attribute and/or controlling an ultrafiltration/diafiltration (UF/DF) pool conditioning process. The method comprises: obtaining, a training dataset comprising: a plurality of Raman measurements of a conditioned UF/DF pool obtained from a Raman spectrometer operationally coupled to a recovery vessel comprising the conditioned UF/DF pool, wherein the recovery vessel is downstream from an ultrafiltration/diafiltration (UF/DF) operation and receives the UF/DF pool for conditioning with a buffer; and corresponding measurements of one or more product quality attributes of the conditioned UF/DF pool. The method further comprises: training a machine learning model, using said training dataset, to predict the one or more product quality attributes of a conditioned UF/DF pool using as input a Raman measurement of the conditioned UF/DF pool. The methods according to the present aspect may have any of the features described in relation to the first or second aspects.

In various embodiments, according to a fifth aspect, there is provided a system comprising: one or more processors; and one or more computer-readable media storing instructions. The instruction, when executed by the one or more processors, cause the one or more processors to execute any of the methods described herein.

In various embodiments, according to a fifth aspect, there is provided one or more non-transitory computer-readable media storing instructions that, when executed by one or more processors, cause the one or more processors to execute any of the methods described herein.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a schematic workflow for real-time (e.g., online) monitoring and control of a conditioned or diluted ultrafiltration/diafiltration (UF/DF) pool, in accordance with various embodiments.

FIG. 2 is a block diagram of a product quality attribute prediction system, in accordance with non-limiting examples of the present disclosure.

FIG. 3 is a flowchart of a machine learning model-based process for predicting product quality attributes of a conditioned or diluted UF/DF pool, in accordance with non-limiting examples of the present disclosure.

FIG. 4 is a flowchart of a machine learning model-based process for controlling a UF/DF pool, in accordance with non-limiting examples of the present disclosure.

FIG. 5 shows two categories of experiments and analyses conducted to develop models for real-time monitoring and control of UF/DF conditioning process and target conditions, in accordance with non-limiting examples of the present disclosure.

FIG. 6A shows a plot of Raman spectra for conditioned UF/DF pool end-point with varying levels of osmolality in a preliminary scale-up analysis, in accordance with non-limiting examples of the present disclosure.

FIG. 6B shows a scatter plot of actual versus predicted osmolality for conditioned UF/DF pool end-point with the Raman spectra in FIG. 6A as model input, in accordance with non-limiting examples of the present disclosure.

FIG. 7A shows a plot of Raman spectra for conditioned UF/DF pool in a design of experiments (DOE) analysis with varying levels of protein concentration, in accordance with non-limiting examples of the present disclosure.

FIG. 7B shows a scatter plot of actual versus predicted protein concentration for conditioned UF/DF pool in a DOE analysis with the Raman spectra in FIG. 7A as model input, in accordance with non-limiting examples of the present disclosure.

FIG. 8A shows a plot of Raman spectra for conditioned UF/DF pool in a trend analysis with varying levels of protein concentration, in accordance to various embodiments.

FIG. 8B shows scatter plot of actual versus predicted protein concentration for conditioned UF/DF pool in trend analysis with the Raman spectra in FIG. 8A as model input, in accordance with non-limiting examples of the present disclosure.

FIG. 9 shows a scatter plot of actual versus predicted osmolality for conditioned UF/DF pool in a trend analysis with the Raman spectra in FIG. 8A as model input, in accordance with various embodiments.

FIG. 10A shows a scatter plot of actual versus predicted protein concentration for conditioned UF/DF pool when a holistic model, developed using a combined Trend and DOE training datasets, is used to predict for end point test data from DOE analysis, in accordance with non-limiting examples of the present disclosure.

FIG. 10B shows a scatter plot of actual versus predicted protein concentration for conditioned UF/DF pool when the holistic model is used to predict for end-to-end test data from Trend analysis, in accordance with non-limiting examples of the present disclosure.

FIG. 11A shows a scatter plot of actual versus predicted protein concentration for conditioned UF/DF pool in trend analysis when a multivariate linear regression model is used, in accordance with non-limiting examples of the present disclosure.

FIG. 11B shows a scatter plot of actual versus predicted protein concentration for conditioned UF/DF pool in trend analysis when a deep neural network model is used, in accordance with non-limiting examples of the present disclosure.

FIG. 12 shows a time-series plot of experiment data comparing real-time protein concentration predictions over time using a model trained with trend analytics data illustrated FIG. 8A, (“process trend model”). The time-series plot for the protein concentration predictions are overlaid by measurements of protein concentration of samples taken throughout the experiment at various time points as will be described herein, and measured using a protein concentration sensor, in accordance with non-limiting examples of the present disclosure.

FIG. 13 shows a time-series plot of experiment data comparing osmolality predictions using Raman spectra collected from real-time experiment in FIG. 12 as input to process trend model versus osmolality of samples taken throughout the experiment and measured using osmometer, in accordance with non-limiting examples of the present disclosure. The experiments pertaining to FIGS. 12 and 13 demonstrate the disclosed monitoring of a UF/DF pool conducted using conditions shown in Table 3 (described further below), where the first step corresponds to the filling up of the recovery vessel with the UF/DF diluted pool (mAbs feedstock), and each additional step represents a subsequent dilution with a conditioning buffer (20 mM Sodium Acetate, 1057 mM (40%) Trehalose, 0.20% w/v Polysorbate 20, pH 5.3).

FIG. 14 shows scatter plot of actual (true measurement) versus predicted (averaged in a window of last five measurements) protein concentration corresponding to the time-series plot in FIG. 12, in accordance with non-limiting examples of the present disclosure.

FIG. 15 shows a scatter plot of actual versus predicted (averaged in a window of last five measurements) osmolality corresponding to time-series plot in FIG. 13, in accordance with non-limiting examples of the present disclosure.

FIG. 16 shows a plot illustrating real-time control of a conditioned UF/DF pool with target protein concentration=50 mg/mL in a recovery vessel based on real-time regulation of the weight of the recovery vessel using feed flow-rate as control variable, in accordance with non-limiting examples of the present disclosure.

FIG. 17 shows a plot illustrating real-time control of a conditioned UF/DF pool with target protein concentration=45 mg/mL in a recovery vessel based on real-time regulation of the weight of the recovery vessel using feed flow-rate as control variable, in accordance with non-limiting examples of the present disclosure.

FIG. 18 is a block diagram of a computer system in accordance with non-limiting examples of the present disclosure.

FIG. 19 illustrates an example neural network that can be used to implement a deep learning neural network in accordance with non-limiting examples of the present disclosure.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Antibodies are protective proteins produced by the immune system in response to the presence of a foreign substance, called an antigen. Antibodies may include monoclonal antibodies (mAbs), polyclonal antibodies (pAbs), antibody fragments (e.g., Fab′, Fab, F(ab′)2), single domain antibodies (DABs), Fv, scFv (single chain Fv), and the like. Monoclonal antibodies (mAbs) are prevalent for both therapeutic uses and research purposes. A number of improvements in large scale manufacturing processes have enhanced the quality of the production of large quantities of mAbs. Efficient recovery and purification of mAbs from cell culture media is an important aspect of the production process. At least for clinical antibodies, the purification process must produce mAbs that are safe for use in human patients, and in a reliable manner. This includes monitoring product quality attributes that include protein attributes (e.g., concentration) and impurities, including host cell proteins, DNA, viruses, endotoxins, aggregates, concentrations, excipients, and other species that have the potential to impact patient safety, efficacy, or potency. One or more of these parameters, including for process intermediates, can be critical process parameters for unit operation performance. These parameters need to be monitored throughout production, and mAb products should be tested at various stages of the downstream processing to ensure that acceptable levels of impurities are present.

Quality control may be accomplished in antibody production processes by analyzing intermediates and formulated drug substance samples. An example of such samples includes a sample of the ultrafiltration/diafiltration (UF/DF) conditioned pool containing drug substance after purification processes in a UF/DF skid or tank of the antibody production system. Quality control of samples can be performed using offline methods for each lot production. For example, samples may be removed from the UF/DF tank and be subjected to offline tests to measure product quality attributes. In some instances, samples may be monitored inline, i.e., at the location of the UF/DF process, and analyzed. For example, Raman spectroscopy may be utilized to monitor the UF/DF process, as discussed in US. Patent Publication No. 2020/062802, titled “Use of Raman Spectroscopy in Downstream Purification,” which is incorporated by reference herein in its entirety.

Once a UF/DF batch undergoes purification in the UF/DF process, the UF/DF bulk will be transferred to a recovery vessel for dilution and conditioning purposes. During the recovery process, UF/DF pools are conditioned with different buffers (e.g., conditioning buffers, dilution buffers, etc.). The drug substance quality attributes at the end of conditioning process are important metrics in order to decide on the release of the drug substance batch, i.e., off-target quality attributes can result in the discard of a batch. The present disclosure provides solutions for automated real-time monitoring and control of the conditioned and/or diluted UF/DF pool in a recovery vessel to guarantee real-time on-target control of quality attributes and avoid process deviations. Additionally, since the measured quality attributes at the end of the conditioning phase (referred to herein as “end point”) are used to decide on the release of the drug substance batch, the final measurement of the quality attributes at the end of the conditioning (referred to herein as “target conditions”), obtained from real-time model predictions, can be used to make instant automated release test decisions, replacing or augmenting the existing manual quality control methods. Alternative manual and offline measurements of product quality attributes can be slow, inefficient, and prone to cause significant product release delays.

The present disclosure provides processes and systems related to measurement of product quality attributes of a conditioned or diluted UF/DF pool in a recovery vessel by utilizing Raman spectroscopy as an inline probe in the recovery vessel. Examples of product quality attributes that can be monitored and controlled by the techniques disclosed herein include color, clarity/opalescence, physical state, pH, osmolality, polysorbate 20 concentration, one or more protein concentrations of the conditioned UF/DF pool, methionine concentration, N-acetyl tryptophan concentration, high molecular weight forms, low molecular weight forms, acidic region, basic region, high mannose, afucosylation, glactosylation, oxidation, microbiological purity, bacterial endotoxin, potency, and/or the like, of the media and molecules (e.g., mAbs) in the recovery vessel. In particular, the present disclosure discusses techniques that make use of inline Raman spectrometer to obtain Raman spectral data of conditioned UF/DF in a recovery vessel, and predict product quality attributes, such as osmolality and protein concentration of the conditioned UF/DF pool using a machine learning model that is trained to intake Raman measurements as input data and predict product quality attributes based on an analysis of the input data. The recovery operations including the conditioning of the UF/DF pool in the recovery vessel can then be controlled in real-time based on the predicted product quality attributes. Additionally, the predicted quality attributes can replace or augment existing manual off-line quality control tests to allow real-time release testing of drugs, such as mAbs and other molecules.

### II. Definitions

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “about” refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to #15%, +10%, +5%, or #1% as understood by a person of skill in the art.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “machine learning” may include the practice of using models to parse data, learn from it, and then make a determination or prediction about something in the world. Machine learning uses models that can learn from data without relying on rules-based programming.

Embodiments of the present disclosure relate to UF/DF pools that comprise an antibody. The term “antibody” as used herein refers to any immunologic binding agent such as IgG, IgM, IgA, IgD and IgE and also refers to any antibody-like molecule that has an antigen binding region, and includes antibody fragments such as Fab′, Fab, F(ab′)₂, single domain antibodies (DABs), Fv, scFv (single chain Fv), and the like. The antibody may be monoclonal or humanized, in specific embodiments.

The term “vessel” as used herein refers to an apparatus suitable for housing and growing a cell culture, including on a manufacturing scale. Further, the term can also refer to a recovery vessel. A recovery vessel is an apparatus suitable for housing a UF/DF pool for a recovery process of diluting and conditioning UF/DF pools with dilution and conditioning buffers, respectively.

The term “conditioning,” as used herein refers to adding dilution and/or conditioning buffers to a solution or media, such as to a UF/DF pool.

The term “cell culture” as used herein refers to growth of cells in an artificial environment under suitable conditions.

The term “molecule” as used herein refers to substances that are produced by cells and may include carbohydrates, lipids, nucleic acids, and proteins. The terms “molecule” and “biomolecule” may be used interchangeably.

As used herein, an “artificial neural network” or “neural network” (NN) may refer to mathematical models or computational algorithms for the training and/or deployment of mathematical models (i.e. for making predictions). Neural networks, which may also be referred to as neural nets, can employ one or more layers of linear or nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may receive training data (learning examples) and learn how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “bioprocess” (also referred to herein as “biomanufacturing process”) refers to a process where biological components such as cells, parts thereof such as organelles or multicellular structures such as organoids or spheroids are maintained in a liquid medium in an artificial environment such as a bioreactor. In embodiments, the bioprocess refers to a cell culture. A bioprocess typically results in a product, which can include biomass and/or one or more compounds that are produced as a result of the activity of the biological components.

As used herein, a “target condition” or a “target matrix condition” refers to a state, a moment, or a phase of aa bioprocess of the UF/DF pool at which product quality attributes is targeted to be monitored, predicted, or controlled. The state, moment, or phase may be predefined or fixed. Thus, a “fixed target matrix condition” may refer to a fixed or predefined moment of the bioprocess (e.g., a recovery phase of the UF/DF pools), at which product quality attributes can be measured or predicted. Such predefined moments include but are not limited to the beginning of the recovery phase (e.g., right after or soon after a UF/DF pool is received at a recovery vessel from a UF/DF tank for recovery), at the end of the recovery phase (e.g., after recovery operations of the conditioned UF/DF pool is completed), or any other moment of the bioprocess.

As used herein, an “end point” refers to a timepoint towards the end of a bioprocess at which a targeted outcome of a bioprocess is analyzed to help determine the efficacy of the product developed via the bioprocess.

As used herein, a “product quality attribute” refers to a physical and/or chemical property of a product comprising one or more biomolecules that are the product of a bioprocess, in solution.

As used herein, a “UF/DF pool” refers to a solution comprising one or more biomolecules that are the product of a bioprocess, where the solution has been obtained by applying ultrafiltration and diafiltration to the output of a bioprocess.

As used herein, a “Raman measurement” may refer to the Raman spectral data obtained by a Raman spectrometer by virtue of detecting the inelastic Raman scattering from a solution (e.g., the conditioned UD/DF pool). The Raman measurement may be used as a data input for the training and/or application of various machine learning models described herein. For example, the Raman measurement may be in the form of image data of a spectrum, a series of peaks in the spectrum, as a data series with an intensity for each wavenumber with a given stride between min and max wavenumbers in the spectrum, or a combination thereof.

As used herein, a “design of experiments (DOE) analysis” refers to analysis of a plurality of measurements of product quality attributes (e.g., protein concentrations, osmolalities, etc.) over a predetermined range using the target matrix conditions at a predefined moment of the bioprocess (e.g., an end of UF/DF pool conditioning process). For example, the predetermined range may encompass all product quality attributes (e.g., protein concentrations) expected at the predefined moment (e.g., end point) of the bioprocess (e.g., a particular UF/DF process). A design of experiments (DOE) partial least squares (PLS) regression model (also referred to herein as a “DOE PLS model”), refers to a PLS regression based machine learning model that is trained using training data comprising the plurality of measurements of the product quality attributes over the DOE analysis, in addition to corresponding Raman measurements taken for the conditioned UF/DF pool at that predefined moment.

As used herein, a “trend analysis (TA)” refers to analysis of a range of measurements of the product quality attributes (e.g., protein concentrations, osmolalities, etc.) from the beginning to the end of a bioprocess (“end-to-end”). For example, for a UF/DF conditioning process that commences with a diluted pool at time t_0 and terminates at time t_final with a conditioned pool, a TA PLS model may be trained using training data comprising a range of protein concentrations (e.g., including the initial protein concentration at time t_0, the final protein concentration at time t_final, and additional protein concentrations therebetween of the UF/DF pool under dilution or conditioning) from the beginning to the end of conditioning phase. A trend analysis (TA) partial least squares (PLS) regression model (also referred to herein as a “TA PLS model”), refers to PLS regression based machine learning model that is trained using training data comprising the range of measurements of the product quality attributes (e.g., protein concentrations, osmolalities, etc.) from the beginning to the end (“end-to-end”) of the UF/DF conditioning process along with the corresponding Raman measurements taken for the conditioned UF/DF pool from end to end.

### III. Real-Time Monitoring and Control of Conditioned or Diluted UF/DF Pool in a Recovery Vessel

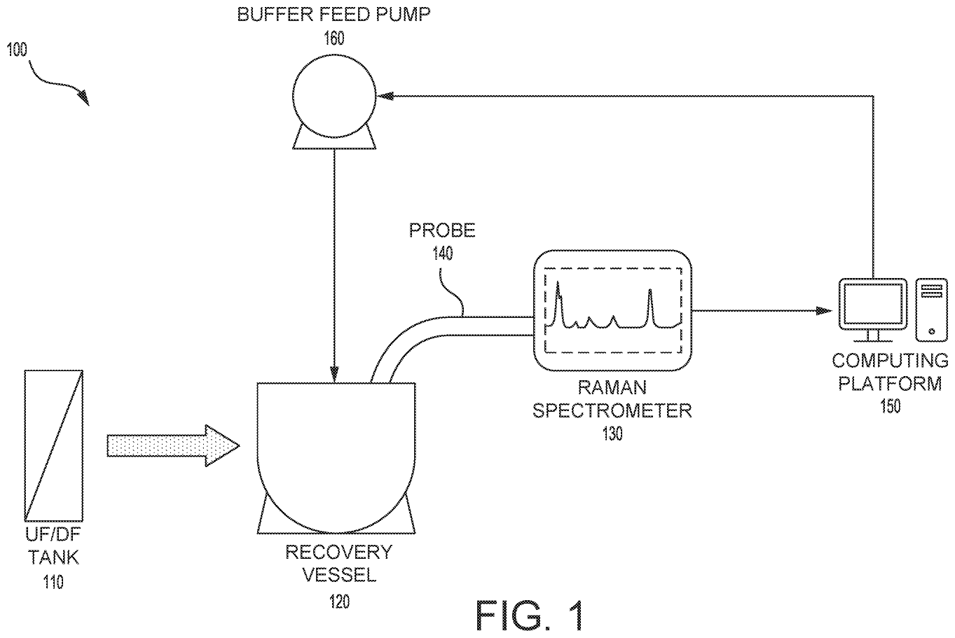

FIG. 1 is a schematic workflow 100 for real-time monitoring and control of a conditioned UF/DF pool in accordance with various embodiments. As discussed above, a UF/DF pool purified at the UF/DF tank 110 may be received at the recovery vessel 120 for dilution or conditioning with buffers to produce the final formulation of the product (e.g., therapeutic drug substance). For example, cell culture media configured to produce monoclonal antibodies may undergo a UF/DF operation or process at the UF/DF tank 110 to remove impurities, reduce volume, and in general achieve conditions that are favorable for therapeutic drug development and storage. The UF/DF pool from the UF/DF tank 110 may then be transferred to the recovery vessel 120 for dilution and conditioning with buffers to form the final formulation of the product (e.g., the monoclonal antibody therapeutics).

Currently, the quality of the drug substances at the recovery vessel 120 are evaluated via several manual steps that include the sampling of the conditioned or diluted UF/DF pool from the recovery vessel 120 to perform off-line measurements of product quality attributes of the conditioned UF/DF pool. Such manual samplings and off-line measurements, however, can be too slow to allow real-time corrections to manufacturing processes and procedures as well as allow instant release of the product. For instance, calculations of bolus additions into a recovery vessel 120 based on the off-line measurements can be too slow for an ongoing therapeutic drug production process and any manual and/or offline corrective actions ensuing from that may be too slow and far from real-time resulting in process deviations and delayed product release.

In various embodiments, a Raman spectrometer 130 can be used to perform inline Raman spectroscopy measurements of the conditioned UF/DF pool in the recovery vessel 120 for real-time monitoring and control of the conditioned UF/DF pool. Inline Raman spectroscopy in the recovery vessel 120 refers to the use of an inline or in-situ probe 140 in the recovery vessel 120 to perform Raman spectroscopy measurements. In such cases, the probe 140 detects the energy of photons inelastically scattered by analytes or molecules of the conditioned UF/DF pool. The photon energies are related to the multiple vibrational modes of the analytes or molecules in the UF/DF pool, which the Raman spectrometer 130 can analyze to generate the Raman spectral data corresponding to the conditioned UF/DF pool. Inline Raman spectroscopy is advantageous in that it is a non-invasive and non-destructive technique that allows for the automated real-time monitoring and control of the conditioned UF/DF pool (e.g., by allowing evaluation of product quality attributes based on the Raman measurements, as discussed in more details below), without the need for the sampling of the conditioned UF/DF pool. In various embodiments, the term “Raman measurements” may refer to the Raman spectral data obtained by the Raman spectrometer 130 by virtue of detecting the inelastic Raman scattering from the conditioned UD/DF pool in the recovery vessel 120. A Raman measurement may be used as a data input for the training and/or application of various machine learning models described herein. For example, the Raman measurement may be in the form of image data of a spectrum (e.g., a full spectrum), a series of peaks in the spectrum, as a data series with an intensity for each wavenumber with a given stride between min and max wavenumbers in the spectrum, or a combination thereof. The use of Raman spectroscopy in characterizing therapeutics is discussed in “Multi-attribute Raman spectroscopy (MARS) for monitoring product quality attributes in formulated monoclonal antibody therapeutics,” by B. Wei et al., mAbs, 14:1, 2007564 (2022), which is incorporated herein by reference in its entirety.

After obtaining the Raman measurements of the conditioned UF/DF pool in the recovery vessel 120 by the Raman spectrometer 130, in various embodiments, the Raman spectrometer 130 transmits the Raman measurements to a computing platform 150 (e.g., at a processor of the computing platform 150) via a communication module that is operationally coupled to the Raman spectrometer 130 and the computing platform. In some instances, the computing platform 150 can be on-site, i.e., at the site where the manufacturing process for producing the therapeutic drug substance is occurring (e.g., the site where UF/DF tank 110 or recovery vessel 120 is located). In some instances, the computing platform 150 can be off-site or remote. For example, a remote computing platform 150 may be accessible to the Raman spectrometer 130 via a wireless network. In some instances, the computing platform can be an edge node (e.g., IOT edge node), an on-premise virtual machine, a cloud server, a cloud serverless solution, or combination thereof.

In various embodiments, the computing platform 150 hosts a machine learning model that may be trained to process the Raman measurements of the conditioned UF/DF pool received from the Raman spectrometer 130 and predict one or more product quality attributes of the conditioned UF/DF pool. For example, the machine learning model can be a multivariate linear model, a neural network model, a deep learning model, an ensemble model, a regression model, or combination thereof. The product quality attributes of the conditioned UF/DF pool that may be predicted by the machine learning model include but are not limited to the protein concentration, osmolality, etc., of the conditioned UF/DF pool.

In some instances, the machine learning model is trained to predict product quality attributes using a training dataset that includes inline Raman measurements of conditioned UF/DF pools and product quality attributes associated therewith (i.e., of these conditioned UF/DF pools) that are measured off-line or in real-time. For example, the training dataset may include product quality attributes of conditioned UF/DF pools measured using offline methods, for example, by applying measurement instruments (e.g., osmometers, protein sensors, etc.) to measure the product quality attributes on samples of conditioned UF/DF pools taken out of the respective conditioned UF/DF pools. Also or alternatively, the training dataset may include product quality attributes for conditioned UF/DF pools measured inline, for example, through measuring instruments (e.g., osmometers, protein sensors, etc.) located near, within, or at the site of the respective conditioned UF/DF pools (e.g., as they are undergoing conditioning), such that samples need not be taken out of the respective conditioned UF/DF pools, or need not be taken out of the conditioning process, for measurement of any product quality attribute. The training dataset may further include the respective Raman measurements for such conditioned UF/DF pools. The training dataset is provided to the machine learning model so that the machine learning model learns to process the inline or offline Raman measurements and be capable of predicting the product quality attributes based on the analysis. In some instances, the training dataset may be a design-of-experiments (DOE) dataset of inline Raman measurements and associated product quality attributes of the conditioned UF/DF pools obtained for training a machine learning model to predict product quality attributes at target matrix conditions (e.g., at the end point of the conditioning or recovery phase of the UF/DF pools in the recovery vessel). DOE is a systematic statistical method for analyzing and quantifying effects and interactions of factors that influence an output, and as such can be applied at data collection stage to arrive at a valid output. In other instances, the training dataset may be a trend-analysis dataset (also referred to herein as “trend analytics dataset”) of inline Raman measurements and associated product quality attributes of the conditioned UF/DF pools obtained for training a machine learning model to predict product quality attributes for beginning-to-end matrix conditions (e.g., from the beginning to the end of the conditioning of the UF/DF pools in the recovery vessel).

As noted above, the Raman spectrometer 130 may use a communications module that is operationally coupled to the computing platform 150 to transmit the Raman measurements of the UF/DF pool in the recovery vessel to the computing platform 150. Upon receiving the Raman measurements, in various embodiments, the computing platform 150 may execute a trained machine learning model hosted thereon to analyze the Raman measurements and predict the product quality attributes of the conditioned UF/DF pool based on the analysis. For example, the trained machine learning model may analyze the Raman measurements and predict product quality attributes of the conditioned UF/DF pool such as but not limited to the protein concentration, osmolality, etc., of the conditioned UF/DF pool.

In various embodiments, the monitoring and control of the conditioned UF/DF pool in the recovery vessel 120 includes the addition, or cessation thereof, of a buffer into the recovery vessel 120 to adjust the product quality attributes of the conditioned UF/DF pool to values that are indicative of target product quality. Also or alternatively, the monitoring and control of the conditioned UF/DF pool in the recovery vessel 120 may further include changing the flow rate of the buffer into the recovery vessel 120, preventing a next scheduled bolus addition of the buffer, momentarily stopping a continuous flow of the buffer, triggering the next bolus addition of the buffer, and the link. That is, for example, the conditioning process of the UF/DF pool in the recovery vessel 120 may cause amounts and/or concentrations of buffers in the recovery vessel 120 to change over time (e.g., via the addition, cessation, altered flow rate, or altered bolus schedule of buffers) to condition or dilute (or cease to dilute or condition) the UF/DF pool received from the UF/DF tank 110. For instance, a first product quality attribute of the conditioned UF/DF pool predicted by the machine learning model can be indicative of product quality at the end point of the conditioning process. A buffer (e.g., a conditioning or dilution buffer) can be next added into the recovery vessel 120 (or the addition terminated if already in progress) as part of the UF/DF conditioning process to achieve a second product quality attribute of the UF/DF pool that is indicative of the target product quality. Alternatively, a first product quality attribute of the conditioned UF/DF pool predicted by the machine learning model can be indicative of product quality at a current point of the dilution process. In such aspects, responsive to a determination that the predicted first product quality attribute at the current point not being a desired or targeted product quality attribute, the UF/DF pool may be further conditioned (e.g., via a conditioning or dilution buffer) as part of the UF/DF conditioning process to achieve a second product quality attribute of the UF/DF pool that is indicative of the target product quality attribute.

In such embodiments, the computing platform 150 may determine the amount of buffer that may needed to be added into the recovery vessel 120 for the conditioned UF/DF pool to achieve the second or target product quality attribute. Alternatively, the computing platform 150 may determine whether an on-going addition of the buffer should be terminated so that the conditioned UF/DF pool achieves, or does not significantly deviate from, the second or target product quality attribute. For example, the computing platform 150 may determine a weight indicator (e.g., weight of the recovery vessel 120, height and/or volume of the conditioned UF/DF pool in the recovery vessel 120, etc.) of the recovery vessel 120 that corresponds to second product quality attribute and compute the amount of buffer that may be needed to be added into the recovery vessel 120 so that the recovery vessel 120 attains the determined weight indicator (e.g., or whether addition of the buffer should be terminated).

As a non-limiting illustrative example, the first product quality attribute predicted by the machine learning model can be a first protein concentration or osmolality of the conditioned UF/DF pool that is indicative of product quality at a given timepoint (e.g., current, beginning, middle, or end point) during the conditioning process. Further, the first product quality attribute (e.g., the first protein concentration or osmolality) can be higher or lower than a second respective product quality attribute (e.g., a second respective protein concentration or osmolality) that is associated with the product having a target product quality. For example, a second product quality attribute (e.g., a second protein concentration or osmolality) can be a second value (a desired or targeted value) of the same attribute of product quality (e.g., protein concentration, osmolality, etc.). In such cases, the computing platform 150 may determine the weight of the recovery vessel 120 that corresponds to the second product quality attribute (e.g., the second protein concentration or osmolality), for instance, based on a pre-determined formula or a table relating the product quality attribute to the weight indicator (e.g., relating protein concentration or osmolality to weight of the recovery vessel 120 in this example). The computing platform 150 may then determine or compute the amount of buffer that needs to be added into the recovery vessel 120 so that the weight of the recovery vessel 120 increases to the determined weight. For instance, the computing platform may have information on the weight of the recovery vessel 120 that corresponds to the first product quality attribute (e.g., the first protein concentration or osmolality) (“first weight”). In such cases, the amount of the buffer that is to be added to the recovery vessel 120 weighs at least substantially equal to the difference between the determined weight and the first weight. In other words, the buffer to be added would increase the weight of the recovery vessel 120 from the first weight to the determined weight.

In such embodiments, the computing platform 150 may output an indication of whether to add or cease adding the buffer into the recovery vessel based on the computed weight indicator of the recovery vessel. For example, the computing platform 150 may generate a buffer pump signal and transmit the buffer pump signal to a buffer feed pump 160 that is configured to pump buffer into the recovery vessel 120 (e.g., from a buffer reservoir). The buffer pump signal may be configured to instruct the buffer feed pump 160 to add buffer into the recovery vessel 120 in an amount that results in the recovery vessel 120 attaining the weight indicator that corresponds to the second product quality attribute (alternatively, the buffer pump signal may instruct the buffer feed pump 160 to cease adding the buffer so the weight indictor of the recovery vessel 120 does not deviate significantly from the determined weight indicator).

In various embodiments, the computing platform 150 may be configured to generate an indication based on the predicted product quality indicating that the product is ready for instant release (e.g., without further manual quality control processes). For example, a machine learning model may be used to predict (e.g., during any given time of a bioprocess), the product quality attribute (e.g., protein concentration, osmolality, etc.) of a UF/DF pool in a recovery vessel. The computing platform 150 may then generate an indication based on the predicted product quality indicating whether the batch of UF/DF pool in the recovery vessel at the given time is ready for immediate release. That is, the indication may indicate that the batch may not need further product quality testing. For example, the computing platform 150 may compare the predicted product quality attribute with a threshold product quality attribute, and may generate the indication based on the comparison. For instance, the indication may indicate the batch is ready (or not ready) for instant release when the comparison shows that the predicted product quality attribute is within (or outside) a pre-determined range of the threshold product quality attribute. In various embodiments, the generation of the indication may be in real-time, i.e., the indication may be generated during or at the end of the recovery process, thereby reducing delay associated with manual product quality testing.

In various embodiments, the monitoring and control of the conditioning of a UF/DF pool in the recovery vessel 120 as depicted in workflow 100 may occur in real-time or near real-time, and without the need for manual sampling of the UF/DF pool from the recovery vessel 120. That is, the processes from the arrival of the UF/DF pool at the recovery vessel 120 to the addition, or termination thereof, of buffer into the recovery vessel 120 by the buffer feed pump 160, inclusive, may occur automatically in real-time or near real-time without manual control or need for manual sampling to, for instance, determine the product quality attributes of the UF/DF pool in the recovery vessel 120. In some instances, the time duration between the transmission of the Raman measurements by the Raman spectrometer 130 to the computing platform 150 and the arrival of the buffer pump signal at the buffer feed pump can be in the range from about 0.1 second to about 3 seconds, from about 0.5 second to about 2.5 seconds, from about 1 second to about 2 seconds, from about 1 second to about 1.5 seconds, from about 1.5 second to about 2 seconds, about 1 second, about 2 seconds, including values and subranges therebetween as well as ranges that combine any of the lower and upper boundaries specified.

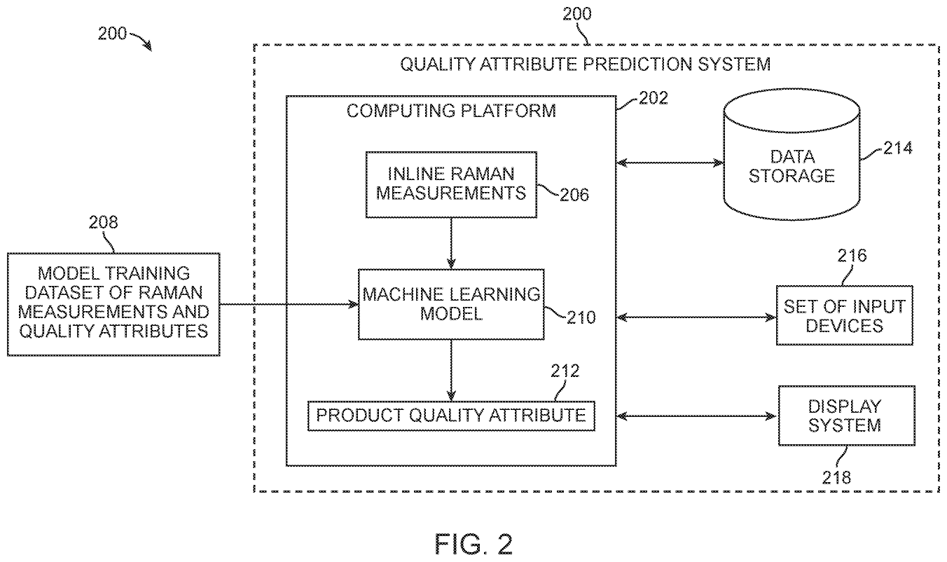

FIG. 2 is a block diagram of a product quality attribute prediction system 200 in accordance with various embodiments. In various embodiments, the quality attribute prediction system 200 may correspond to the computing platform 150 of FIG. 1. For example, the quality attribute prediction system 200 can be an edge node (e.g., IOT edge node), a virtual machine (e.g., an on-premise virtual machine), a cloud server, a cloud serverless solution, or combination thereof, that is in remote communication with the recovery vessel of a conditioned UF/DF pool. Quality attribute prediction system 200 uses a machine learning model 210 to predict a product quality attribute of a conditioned UF/DF pool in a recovery vessel. An example of the product quality attribute includes osmolality or protein concentration of the conditioned UF/DF pool. In some instances, a UF/DF pool may be received at the recovery vessel from a UF/DF tank, and the UF/DF pool may be conditioned with a buffer to assist with a recovery operation to recover the drug substance (e.g., mAbs) therein. In such cases, inline Raman measurements 206 of the UF/DF pool may be received at the quality attribute prediction system 200 so that the machine learning model 210, trained on a training dataset 208 of Raman measurements and product quality attributes associated thereto to predict the product quality attributes, can predict the product quality attributes 212 of the conditioned UF/DF pool. Quality attribute prediction system 200 includes computing platform 202, data storage 214, set of input devices 216, and display system 204.

Computing platform 202 may take various forms. In various embodiments, computing platform 202 includes a single computer (or computer system) or multiple computers in communication with each other. In other examples, computing platform 202 takes the form of a cloud computing platform. In various embodiments, computing platform 202 may be communicatively coupled with data storage 214, set of input devices 216, display system 204, or a combination thereof. In various embodiments, data storage 214, set of input devices 216, display system 204, or a combination thereof may be considered part of or otherwise integrated with computing platform 202. Thus, in some examples, computing platform 202, data storage 214, set of input devices 216, and display system 204 may be separate components in communication with each other, but in other examples, some combination of these components may be integrated together.

In various embodiments, a Raman spectrometer performs Raman scans of the conditioned UF/DF pool in the recovery vessel to generate the inline Raman measurements 206. The inline Raman measurements 206 can be Raman spectral data of the conditioned UF/DF pool. For example, the Raman spectrometer (e.g., such as Raman RXN2 spectrometer from Kaiser Optical Systems, Inc) can use an inline probe that is operationally coupled to the recovery vessel to perform one or more Raman scans of the conditioned UF/DF pool at a desired rate (e.g., a scan every few minutes). The Raman scans can be performed over a wide range of wavenumbers (e.g., from about 100/cm to about 3000/cm), and the Raman spectrometer can then generate a Raman spectral data of the UD/DF pool based on the Raman scans, where the Raman spectral data includes peaks at those wavenumbers corresponding to Raman shifts (e.g., those wavenumbers at which inelastic scattering of photons by components of the conditioned UF/DF pool occurs).

In various embodiments, the inline Raman measurements 206 are provided to the machine learning model 210 for analysis and use in predicting the product quality attributes of the conditioned UF/DF pool. In some instances, additional data related to the conditioned UF/DF pool or the recovery vessel containing the conditioned UF/DF pool may also be provided to the machine learning model 210. For example, a pH probe may be operationally coupled to the recovery vessel and may measure the pH of the conditioned UF/DF pool (e.g., and store such measurements in the data storage 214). As another example, a temperature probe may be operationally coupled to the recovery vessel and may measure the temperature of the conditioned UF/DF pool or the recovery vessel (e.g., and store such measurements in the data storage 214). As such, the additional data related to the conditioned UF/DF pool or the recovery vessel can be the measured pH and/or temperature of the conditioned UF/DF pool, and such data may also be provided to the machine learning model 210 as input data.

The machine learning model 210 can be but is not limited to a neural network, a decision tree, a random forest, a support vector machine, a Bayesian network, a regression model, a multivariate linear model, an ensemble model, etc., or combination thereof. The neural network can be a deep neural network, a convolutional neural network (CNN), an artificial neural network (ANN), a recurrent neural network (RNN), a modular neural network (MNN), a residual neural network (RNN), an ordinary differential equations neural networks (neural-ODE), a squeeze and excitation embedded neural network, a MobileNet, etc. The ANN can be a long short-term memory (LSTM) neural network. The regression model can be a gradient boosting machine (GBM) model (e.g., XGBoost). The regression model can also be a linear regression model (e.g., including multivariate linear regression model), a logistic regression model, a polynomial regression model, a ridge regression model, a least absolute shrinkage and selection operator (LASSO) regression model, a partial least square (PLS) regression model, a principal components regression model, and/or the like. A LASSO regression model is a regularized linear regression model that performs regularization and variable or feature selection to improve the prediction accuracy of the model. LASSO regression uses L1 penalty for both fitting and penalizing of model coefficients, and it performs the variable selection and the regularization simultaneously to improve both prediction accuracy and interpretability of the model simultaneously. PLS methods assume that the input/observed variables generated by a system or process driven by a small number of latent (e.g., not directly observed or measured) variables, and models relationships between the observed variables with the latent variables. The data input for these models may include but are not limited to image data of a spectra (e.g., corresponding to Raman measurements), a series of peaks in the spectra, as a data series with an intensity for each wavenumber with a given stride between min and max wavenumbers in the spectra, etc.

In various embodiments, the machine learning model 210 may be trained with the training dataset 208 of Raman measurements and product quality attributes associated thereto so that the machine learning model 210 is capable of intaking the inline Raman measurements 206 as input data and predicting the product quality attributes 212 of the UF/DF pool. In some instances, the training dataset 208 may be a dataset of Raman measurements and associated product quality attributes of UF/DF pools obtained at fixed target matrix conditions. As used herein, a fixed target matrix condition may refer to a predefined moment of a bioprocess (e.g., the recovery phase of the UF/DF pools), at which product quality attributes can be measured or predicted. Predefined moments include but are not limited to the beginning of the recovery phase (e.g., right after or soon after a UF/DF pool is received at a recovery vessel from a UF/DF tank for recovery), at the end of the recovery phase (e.g., after recovery operations of the conditioned UF/DF pool is completed), any other moment (e.g., duration or instance of time) of the recovery phase, etc. The Raman measurements of the training dataset may be obtained via inline or offline Raman spectroscopy performed on conditioned UF/DF pools at fixed target matrix conditions, and the associated product quality attributes may also be obtained or measured offline or real-time. For example, samples of the conditioned UF/DF pools at a fixed target matrix condition (e.g., end of the recovery phase) may be retrieved and the protein concentration thereof may be measured offline with protein concentration sensors (e.g., SoloVPE System® by C Technologies, Inc™). As another example, an osmometer may be utilized on the samples to measure the osmolality of the UF/DF pools. In such cases, the Raman measurements and the measured product quality attributes (e.g., measured protein concentration, measured osmolality, etc.) may be combined to form the training dataset 208 for that fixed target matrix condition. Such training dataset 208 that includes Raman and product quality attribute measurements of a fixed target matrix condition may be referred to as a design-of-experiment (DOE) dataset. In various embodiments, a machine learning model 210 that is trained using a DOE training dataset may be referred to as a DOE machine learning model.

In some instances, the training dataset 208 may be a dataset of Raman measurements and associated product quality attributes of UF/DF pools obtained for a range of recovery phase matrix conditions. This range of recovery phase matrix conditions may correspond to a selected duration of the recovery phase of the UF/DF pools. For example, the duration can be the entire duration of the recovery phase from beginning (e.g., receiving of the UF/DF pool at the recovery vessel or end of dilution) to the end (e.g., the end of the recovery or conditioning phase of the conditioned UF/DF pool), i.e., the Raman measurements and associated product quality attributes may be obtained for beginning-to-end matrix conditions of the conditioned UF/DF pool recovery operations. In some instances, the selected duration can be shorter than the entire duration of the recovery operation or conditioning phase. Raman measurements and associated product quality attributes of UF/DF pools obtained for a range of recovery phase matrix conditions facilitate the analysis and determination of UF/DF pool behaviors and trends over the duration of the recovery operation. As such, a training dataset 208 that includes such measurements for a range of recovery phase matrix conditions may be referred to as trend analytics (TA) training datasets. In various embodiments, a machine learning model 210 that is trained using a TA training dataset may be referred to as a “TA machine learning model” or “process trend model.”

In various embodiments, the Raman measurements and the associated product quality attributes for a range of recovery phase matrix conditions may be obtained by performing multiple measurements over the desired target matrix conditions. For example, different amounts of buffer may be added to the recovery vessel of a conditioned UF/DF pool over the duration of the recovery operation to arrive at the desired matrix conditions, and the Raman measurements and associated product quality attributes of the UF/DF pools may be obtained after each buffer addition to obtain the Raman measurements and the associated product quality attributes for that range of recovery phase matrix conditions (e.g., beginning-to-end recovery phase matrix conditions). In various instances, the Raman measurements may be obtained via inline Raman spectroscopy performed on the conditioned UF/DF pools. Further, the associated product quality attributes may also be obtained or measured offline. For example, samples of the conditioned UF/DF pools may be retrieved over the range of recovery phase matrix conditions and the protein concentration thereof may be measured offline with protein concentration sensors (e.g., SoloVPE System® by C Technologies, Inc™). As another example, an osmometer may be utilized on the samples to measure the osmolality of the UF/DF pools over the range of recovery phase matrix conditions. In such cases, the Raman measurements and the measured product quality attributes (e.g., measured protein concentration, measured osmolality, etc.) obtained for the range of recovery phase matrix conditions may be combined to form the TA training dataset.

In various embodiments, as discussed above, the inline Raman measurements 206 can be Raman spectroscopy measurements of the UF/DF pool obtained by an inline Raman spectrometer operationally coupled to the recovery vessel of the UF/DF pool that is undergoing a recovery operation (e.g., including conditioning or dilution with buffers). In such cases, one can use the trained machine learning model 210 and the inline Raman measurements 206 to determine the product quality attributes such as but not limited to protein concentration, osmolality, etc., of the conditioned UF/DF pool in real-time or nearly real-time, and do so without necessarily having to retrieve samples of the conditioned UF/DF pool manually. For example, a technician may wish to adjust in real-time the amount of buffer that is being added to the recovery vessel (e.g., to increase or decrease the product quality attributes) to improve yield of valuable molecules (e.g., mAbs) in the recovery vessel, while avoiding product contamination, time- and resource-waste, and other deviations that may be associated with manual product sampling and product attributes determinations.

In such cases, the inline Raman measurements 206 of the conditioned UF/DF pool may be provided to the machine learning model 210 so that the machine learning model 210 analyzes the inline Raman measurements 206 and predicts the product quality attributes 212 of the conditioned UF/DF pool. In some instances, the inline Raman measurements 206 may be obtained for a fixed target matrix conditions, and in such cases, the inline Raman measurements 206 may be provided to the DOE machine learning model for prediction of the product quality attributes of the conditioned UF/DF pool. In some instances, the inline Raman measurements 206 may be obtained for a range of recovery phase matrix conditions (e.g., beginning-to-end recovery phase matrix conditions) and in such cases, the inline Raman measurements 206 may be provided to the TA machine learning model. In such cases, in various embodiments, the buffer used for recovery operations of the conditioned UF/DF pool (e.g., the conditioned UF/DF pool of the inline Raman measurements 206) may be the same as the buffer used for the recovery operations of the UF/DF pools from which the training dataset 208 are obtained.

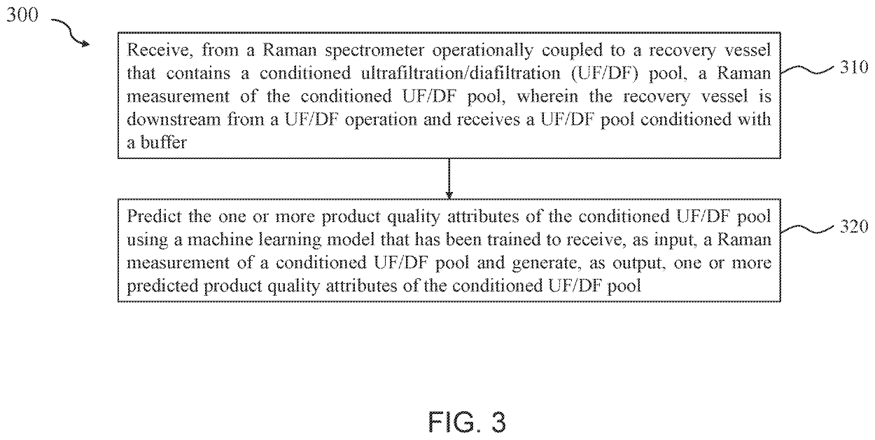

FIG. 3 is a machine learning model-based process 300 for predicting one or more product quality attributes of a bioprocess (e.g., conditioning UF/DF pool), in accordance with various embodiments. In various embodiments, process 300 is implemented using a system, such as, the product quality attribute prediction system 200 of FIG. 2 and/or a processor of the computing platform 150 of FIG. 1 to predict product quality attributes of a conditioned UF/DF pool in a recovery vessel. For example, a UF/DF pool that has undergone purification at a UF/DF skid or tank may be received at a recovery vessel for recovery operations (e.g., which may include conditioning via buffer additions (e.g., conditioning buffers, dilution buffers, etc.)) and a technician may be tasked with monitoring and controlling the recovery operations. For instance, to enhance the yield of the recovery operations (e.g., recovery of valuable molecules such as mAbs in the conditioned UF/DF pool), the technician may wish to adjust the product quality attributes of the conditioned UF/DF pool in real-time without necessarily having to manually retrieve samples of the conditioned UF/DF pool. In such cases, the technician may utilize process 300 to obtain predictions of one or more product quality attributes of the conditioned UF/DF pool (e.g., protein concentration, osmolality, etc.), which can then be used for deciding whether to add, or terminate adding, buffers into the recovery vessel.

At step 310, a processor receives, from a Raman spectrometer operationally coupled to a recovery vessel that contains a conditioned UF/DF pool, a Raman measurement of the conditioned UF/DF pool. The recovery vessel (e.g., recovery vessel 120) is downstream from a UF/DF operation (e.g., occurring at UF/DF tank 110) and receives a UF/DF pool conditioned with a buffer. For example, the processor may receive the real-time Raman measurement of a conditioned UF/DF pool from the Raman spectrometer operationally coupled to the recovery vessel that receives, downstream from the ultrafiltration/diafiltration (UF/DF) operation, a UF/DF pool for conditioning with a buffer. In some instances, the UF/DF pool is a purified UF/DF pool received at the recovery vessel from an upstream filtration vessel after undergoing purification. In some instances, the conditioned UF/DF pool includes an antibody, such as a monoclonal antibody (mAb).

At step 320, the processor predicts the one or more product quality attributes of the conditioned UF/DF pool using a machine learning model that has been trained to receive, as input, a Raman measurement of a conditioned UF/DF pool and generate, as output, one or more predicted product quality attributes of the conditioned UF/DF pool. For example, the processor may apply the real-time Raman measurement to the trained machine learning model to predict a product quality attribute of the conditioned UF/DF pool. In some instances, the machine learning model may have been trained to predict the one or more product quality attributes using a training dataset that includes the one or more product quality attributes of conditioned UF/DF pools conditioned with differing amounts of the buffer added therein and associated real-time Raman measurements of the conditioned UF/DF pools conditioned with the differing amounts of the buffer. In some instances, the trained machine learning model is a trained multivariate linear model, a trained neural network, a trained deep learning model, or a trained ensemble learning model.

In some instances, the one or more product quality attributes include osmolality of the conditioned UF/DF pool. In some instances, the one or more product quality attributes include protein concentration of the conditioned UF/DF pool.

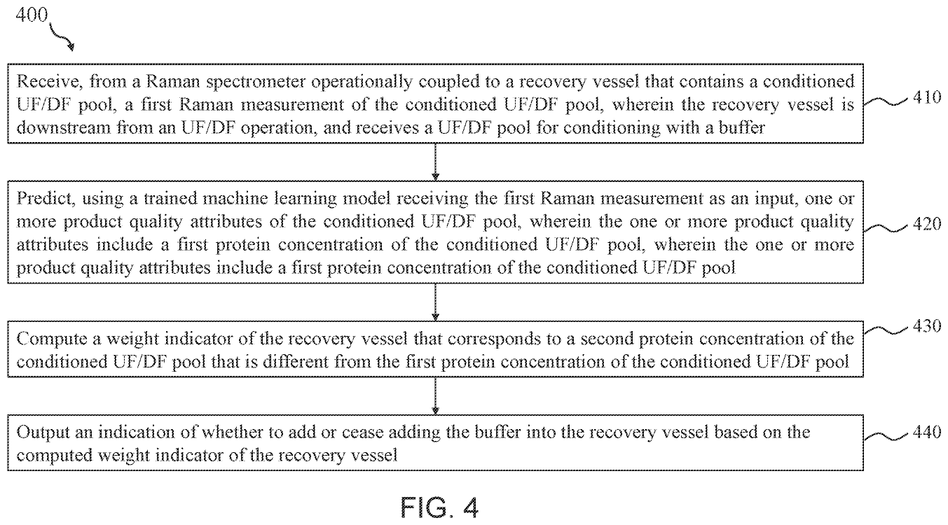

FIG. 4 shows a flowchart of a machine learning model-based process 400 for controlling a UF/DF pool conditioning process (e.g., in a recovery vessel), in accordance with various embodiments. In various embodiments, process 400 is implemented using a system, such as, the product quality attribute prediction system 200 of FIG. 2 and/or the computing platform 150 of FIG. 1 (e.g., a processor of the computing platform 150) to predict one or more product quality attributes (e.g., protein concentration, osmolality, etc.) of a conditioned UF/DF pool in a recovery vessel and generate, based on the predicted product quality attribute, an indication of whether to add or cease adding a buffer into the recovery vessel. For example, the indication may comprise a buffer pump signal to transmit to a buffer pump so that the buffer pump can add, or cease adding, conditioning buffer into the recovery vessel to change the product quality attribute of the conditioned UF/DF pool. The system may determine a weight indicator of the recovery vessel or the UF/DF pool (e.g., weight of recovery vessel including UF/DF pool, volume or height of the UF/DF pool in the recovery vessel, etc.) that corresponds to a new product quality attribute of the UF/DF pool, and may generate a buffer pump signal that causes the buffer pump to add, or cease adding, buffer, such that the recovery vessel or the UF/DF pool attains the weight indicator.

At step 410, a processor of a computing platform receives, from a Raman spectrometer operationally coupled to a recovery vessel that contains a conditioned UF/DF pool, a first Raman measurement of the conditioned UF/DF pool. The recovery vessel is downstream from an UF/DF operation, and receives a UF/DF pool for conditioning with a buffer. For example, the processor may receive the first Raman measurement of the conditioned UF/DF pool from the Raman spectrometer that is operationally coupled to the recovery vessel that receives, downstream from the UF/DF operation, the UF/DF pool for conditioning with the buffer. In some instances, the first Raman measurement is performed in real-time during the conditioning process of the UF/DF pool. In some instances, the computing platform is in remote and/or in wireless communication with the Raman spectrometer and the buffer pump; and a latency between the receiving and the transmitting is no greater than about 2s. For example, the computing platform can be an IOT edge node, an on-premise virtual machine, a cloud server, or a cloud serverless solution. In some instances, the conditioned UF/DF pool includes a monoclonal antibody (mAb).

At step 420, the processor predicts, using a trained machine learning model receiving the first Raman measurement as an input, one or more product quality attributes of the conditioned UF/DF pool. The one or more product quality attributes include a first protein concentration of the conditioned UF/DF pool. For example, the processor may use the trained machine learning model to predict the first protein concentration of the conditioned UF/DF pool based on an analysis of the first Raman measurement. In some instances, the predicting includes predicting an osmolality of the conditioned UF/DF pool based on the analysis of the first Raman measurement. In some instances, the trained machine learning model can be a trained multivariate linear model, a trained neural network, a trained deep learning model, a trained ensemble learning model, or combination thereof.

At step 430, the processor computes a weight indicator of the recovery vessel that corresponds to a second protein concentration of the conditioned UF/DF pool that is different from the first protein concentration of the conditioned UF/DF pool.

At step 440, the processor outputs an indication of whether to add or cease adding the buffer into the recovery vessel based on the computed weight indicator of the recovery vessel. For example, the processor may transmit, to a buffer pump operationally coupled to the recovery vessel, a buffer pump signal configured to instruct the buffer pump to add or cease adding the buffer into the recovery vessel based on the computed weight indicator of the recovery vessel. In some instances, the buffer is added in an amount sufficient to increase the weight of the recovery vessel to within a weight threshold of the computed weight. In various embodiments, the addition of the buffer into the recovery vessel occurs without a sample of the conditioned UF/DF pool being collected from the recovery vessel to measure the first protein concentration. In some instances, responsive to an indication to add the buffer, the buffer may be added in an amount sufficient to increase a weight indicator of the recovery vessel to within a weight threshold of the computed weight indicator.

In various embodiments of method 400, the processor further receives from the Raman spectrometer a second Raman measurement of the conditioned UF/DF pool after the transmitting. In such cases, the processor is configured to predict, using the trained machine learning model taking the second Raman measurement as input, a third protein concentration of the conditioned UF/DF pool. Further, the processor compares the second protein concentration to the third protein concentration to determine effectiveness of the addition of the buffer into the recovery vessel.

In some instances, method 400 does not use any measurement obtained by extracting a sample of the conditioned UF/DF pool from the recovery vessel. For example, the addition of the buffer into the recovery vessel may occur without having to collect or extract a sample of the conditioned UF/DF pool from the recovery vessel to measure (e.g., offline) any product quality attribute (e.g., any protein concentration or osmolality).

### IV. Machine Learning Modeling of UF/DF Quality Attributes Monitoring and Control

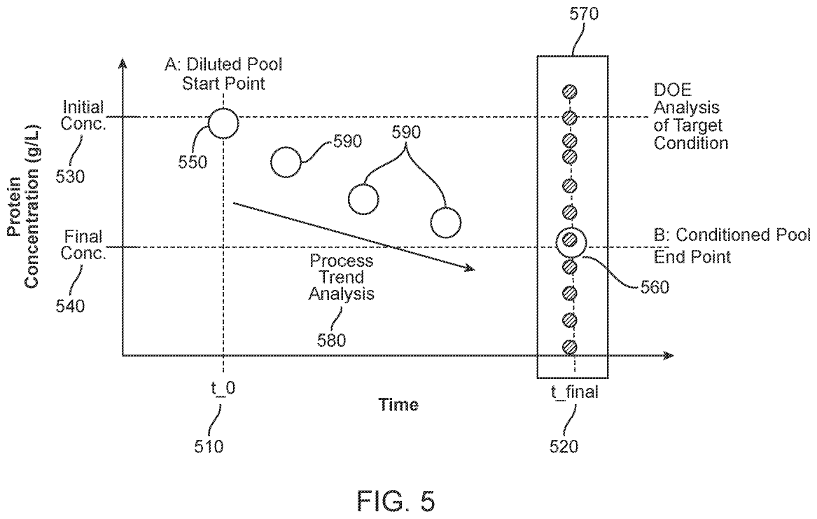

FIGS. 5-11B show figures illustrating the example implementations of the techniques disclosed herein to build (e.g., construct, train, test, etc.) machine learning models that are configured to predict product quality attributes of conditioned UF/DF pools. The built machine learning models were linear partial least squares (PLS) regression models (referred to hereinafter as “PLS models”), deep neural network models, or linear least absolute shrinkage and selection operator (LASSO) models; however, it is to be understood that such regression models were chosen solely for illustrative reasons, and that any of the machine learning models disclosed herein can be built as discussed herein to predict product quality attributes of conditioned UF/DF pools. In a first aspect of the example implementations, it was demonstrated that PLS models trained to predict product quality attributes of small-sized conditioned UF/DF pools (e.g., about 25 mL) were found to accurately predict the product quality attributes of conditioned UF/DF pools that were more than an order of magnitude larger, demonstrating the scalability of the built PLS models. Further, two types of PLS models were built: “DOE PLS models” trained with design of experiments (DOE) training datasets and “TA PLS models” trained with trend analysis (TA) training datasets. It was shown that DOE PLS models were poor at predicting TA test datasets and TA PLS models were poor at predicting DOE test datasets, recommending that the TA PLS models should be used for end-to-end monitoring and control of conditioning phase and the DOE PLS models should be used for tighter control of target conditions to allow real-time release testing of drug substance. Alternatively, it is demonstrated that a holistic model using combined DOE and TA training datasets can be developed to predict quality attributes in both end-to-end and target conditions simultaneously.

FIG. 5 shows two categories of experiments and analyses conducted to develop models for real-time monitoring and control of UF/DF conditioning process and target conditions, in accordance with various embodiments. In various embodiments, a design of experiments (DOE) analysis refers to an analysis conducted to model a plurality of protein concentrations over a predetermined range of concentrations) using the target matrix conditions at the end of UF/DF pool conditioning step or other predefined moment of a bioprocess. In some aspects, the predetermined range may encompass all concentrations expected at the end point of the UF/DF process or other predefined moment of the bioprocess. For example, for a UF/DF conditioning step that commences with a diluted pool 550 at time t_0 510 and is completed at time t_final 520 with a conditioned pool 560, a DOE analysis 570 may refer to an analysis conducted to model the range of protein concentrations (e.g., including the initial protein concentration 530 and the final protein concentration 540) using the target matrix conditions of the range of protein concentrations at the completion of the UF/DF pool conditioning step at t_final 520. In some instances, such experiments may achieve tighter control at target conditions than experiments relying on trend analysis (TA), as discussed below, and may allow real-time release of the product.

In various embodiments, a trend analysis (TA) of the UF/DF conditioning or dilution process refers to an analysis conducted to model full range of protein concentrations from the beginning to the end of conditioning phase (“end-to-end”). For example, for a UF/DF conditioning step that commences with a diluted pool 550 at time t_0 510 and is completed at time t_final 520 with a conditioned pool 560, a process TA 580 may refer to an analysis conducted to model the full range of protein concentrations (e.g., including the initial protein concentration 530, the final protein concentration 540, and additional protein concentrations therebetween of the UF/DF pool 590 under dilution or conditioning) from the beginning 510 to the end 520 of conditioning phase. In some instances, such experiments can achieve real-time monitoring and control of UF/DF conditioning.

The scalability of trained PLS models was demonstrated using a 25 mL conditioned UF/DF pool and a 700 mL conditioned UF/DF pool, where a PLS model was trained using training datasets of Raman and osmolality measurements of the 25 mL conditioned UF/DF pools to predict the osmolality of the 700 mL conditioned UF/DF pools. In setting up the scalability experiment, a combination of two different buffers (modified diafiltration buffer with polysorbate 20 added and modified conditioning buffer with low polysorbate 20) was used to create the samples of conditioned UF/DF pool. Table 1 below lists the trehalose concentration (in mM) and corresponding measured osmolality of the samples.

TABLE 1

| Conditioned UF/DF Pool | Trehalose | Measured Osmolality |
|---|---|---|
| Sample No. | (mM) | (mOsm/kg) |
| 1 | DI Water | NA |
| 2 | 65.7 | 100 |
| 3 | 85.7 | 120 |
| 4 | 105.7 | 142 |
| 5 | 125.7 | 163 |
| 6 | 145.7 | 184 |

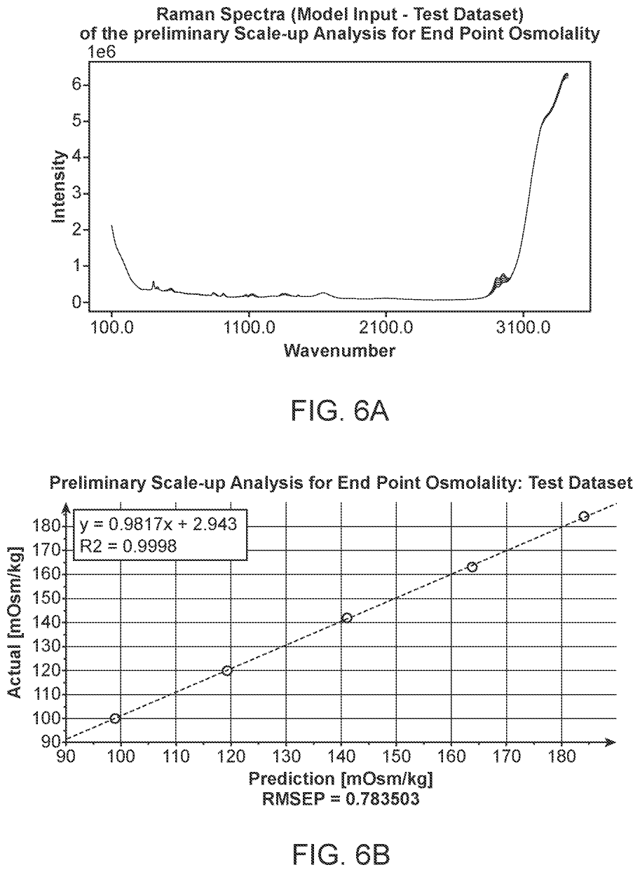

FIG. 6A shows a plot of the Raman scans or measurements collected for the larger 700 mL conditioned UF/DF pool contained in a miniferm. Initially, a recovery vessel was loaded with 25 mL of UF/DF pools and an inline Raman probe was used to collect the Raman scans or measurements. The Raman scans were collected using a Raman spectrometer that was set at exposure of 15 sec, count of 10, and a power of 400 mW. The collection mode was set as periodic with acquisition time of 3 minutes. The recovery vessel was cleaned and sanitized with isopropyl alcohol (IPA) prior to executing the scalability experiment on each sample in Table 1. Then a miniferm was loaded with 700 mL of the UF/DF pool. The miniferm was disassembled, cleaned and sanitized with water and IPA before executing the experiment for each sample in Table 1. The Raman scans, for the larger 700 mL conditioned UF/DF pool and the smaller 25 mL UF/DF pool, were slightly different at wavenumbers below 500 and above 3000. Repeatability was acceptable, as was demonstrated by the similarity of Raman scans (for at least wavenumbers between 500 and 3000) between the larger 700 mL conditioned UF/DF pool and the smaller 25 mL UF/DF pool.

The PLS model was trained using a training dataset of the Raman and osmolality measurements of the 25 mL conditioned UF/DF pool and then was tested using the Raman measurements of the 700 mL conditioned UF/DF pool as input data to predict the osmolality of the 700 mL conditioned UF/DF pool. The osmolality values were measured offline for all the conditioned UF/DF pool samples in Table 1. The PLS model used the 500-1500 wavenumbers, with pretreatment of standard normal variate (SNV) and first derivative (1st derivative) and one latent variables was used using inline Raman scans in the 25 mL vessel. The osmolality predictions of the trained PLS model are shown in FIG. 6B in comparison with the measured osmolality values. The small root mean square error of prediction (RMSEP) and the high correlation coefficient of predictions confirm that the PLS model trained using training datasets collected from the 25 mL conditioned UF/DF pool can predict the osmolality of the 700 mL conditioned UF/DF pool, demonstrating the scalability of trained PLS models.

DOE machine learning models (e.g., including DOE PLS models) and TA machine learning models (e.g., including TA PLS models) were also developed using 25 mL and 700 mL of conditioned UF/DF pools (e.g., same ones as those in the experiment above). Table 2 below shows the experimental design to develop DOE and TA PLS models that are trained to predict protein concentration and osmolality of the conditioned UF/DF pools. The DOE training and testing datasets were obtained from the small scale recovery vessel experiments in conditioned UF/DF pool samples 1-13, which focus on capturing the behavior of a full range of protein concentrations using target matrix conditions (i.e., DOE analysis). In addition to in-line Raman scans, off-line measurements including protein concentration and osmolality and were also collected for all the conditioned UF/DF pool samples. The TA training datasets were obtained from the small recovery vessel experiments in samples 22-29, which focus on capturing the behavior of a full range of protein concentrations using beginning-to-end matrix conditions. Samples 14-21 are samples in the large recovery vessel containing the same material as those in the small recovery vessel of samples 14-21. Thus samples 22-29 and 14-21 have similar chemical properties, although the scales are different (25 mL vs. 700 mL). Furthermore, although the train and test samples were developed independently at different times and dates, both train and test datasets corresponded to the same molecule and had consistent buffer properties in all phases of experiment execution.

TABLE 2

| | | | | Target | Measured | |
|---|---|---|---|---|---|---|
| Conditioned | Small (25 mL)/ | | | Protein | Protein | Measured |
| UF/DF Pool | Large (700 mL) | | Trend/DOE | Concentration | Concentration | Osmolality |
| Sample | recovery vessel | Train/Test | Analysis | (mg/mL) | (mg/mL) | (mOsm/kg) |
| 1 | small | train | DOE | 60 | 60.25241 | 163 |
| 2 | small | train | DOE | 57 | 56.81528 | 162 |
| 3 | small | train | DOE | 53 | 54.02021 | 163 |
| 4 | small | train | DOE | 51 | 52.03757 | 160 |
| 5 | small | train | DOE | 48 | 48.18399 | 162 |
| 6 | small | train | DOE | 45 | 44.80262 | 161 |
| 7 | small | train | DOE | 42 | 41.86378 | 161 |
| 8 | small | test | DOE | 58.5 | 58.64256 | 162 |
| 9 | small | test | DOE | 55.5 | 55.81336 | 161 |
| 10 | small | test | DOE | 52.5 | 52.89516 | 162 |
| 11 | small | test | DOE | 49.5 | 49.61009 | 162 |
| 12 | small | test | DOE | 46.5 | 46.74899 | 161 |
| 13 | small | test | DOE | 43.5 | 43.29222 | 159 |
| 14 | large | test | Trend | 55.6 | 55.74 | 39 |
| 15 | large | test | Trend | 52.5 | 52.50305 | 108 |
| 16 | large | test | Trend | 51.0 | 51.03875 | 142 |
| 17 | large | test | Trend | 49.5 | 49.85859 | 175 |
| 18 | large | test | Trend | 48.0 | 47.89801 | 217 |
| 19 | large | test | Trend | 46.5 | 46.52628 | 251 |
| 20 | large | test | Trend | 45.0 | 44.92265 | 286 |
| 21 | large | test | Trend | 43.5 | 43.29574 | 321 |
| 22 | small | train | Trend | 55.6 | 55.74 | 39 |
| 23 | small | train | Trend | 52.5 | 52.50305 | 108 |
| 24 | small | train | Trend | 51.0 | 51.03875 | 142 |
| 25 | small | train | Trend | 49.5 | 49.85859 | 175 |
| 26 | small | train | Trend | 48.0 | 47.89801 | 217 |
| 27 | small | train | Trend | 46.5 | 46.52628 | 251 |
| 28 | small | train | Trend | 45.0 | 44.92265 | 286 |
| 29 | small | train | Trend | 43.5 | 43.29574 | 321 |

In Table 2, “DOE analysis” refers to experiments corresponding to the final target specifications of the recovery or conditioning phase while “trend analysis” refers to experiments corresponding to full specifications of the recovery or conditioning phase. The DOE samples of conditioned UF/DF pools were made using a combination of two different buffers (modified conditioning buffer with low polysorbate 20 added and a formulation buffer (1 part 10× standard conditioning buffer diluted with 9 parts standard diafiltration buffer) and a monoclonal antibody feedstock. Raman and product quality attribute (e.g., protein concentration and osmolality) measurements for the training dataset of the DOE PLS model were obtained from samples 1-7 and the inline Raman measurements for the testing dataset of the DOE PLS model were obtained from samples 8-13. The “trend” conditioned UF/DF pool samples in the large recovery vessel (samples 14-21), which are used for obtaining the testing dataset for testing the TA PLS model, were made using a combination of two different buffers (10× standard conditioning buffer and diafiltration buffer) and the monoclonal antibody feedstock to reach the conditions shown in Table 2. The training dataset for training the TA PLS model was obtained from samples 22-29, which are samples of about 25-30 mL of conditioned UF/DF pools retrieved from the respective samples 14-21 after Raman scans of the latter were collected. The samples were used to take inline Raman scans in the 25 mL recovery vessel and offline measurements including protein concentration, offline Raman scans and osmolality. To ensure accuracy and reliability for the experiment, five Raman spectra were collected per sample and average via an aggregation method for averaging. The protein concentrations were measured using a protein concentration sensor (SoloVPE System® by C Technologies, Inc™) and the osmomolality was measured using an osmometer.

The amounts of buffer that is needed to transition step by step from sample 14 to 21, or from sample 22 to 29, were calculated, and the buffers in those amount were added into the recovery vessels through a pipette. The impeller was turned on for about 5 minutes to allow the buffer to mix into the UF/DF pools. The same steps were repeated until reaching target protein concentration of 43.5 g/L as shown in Table 2.

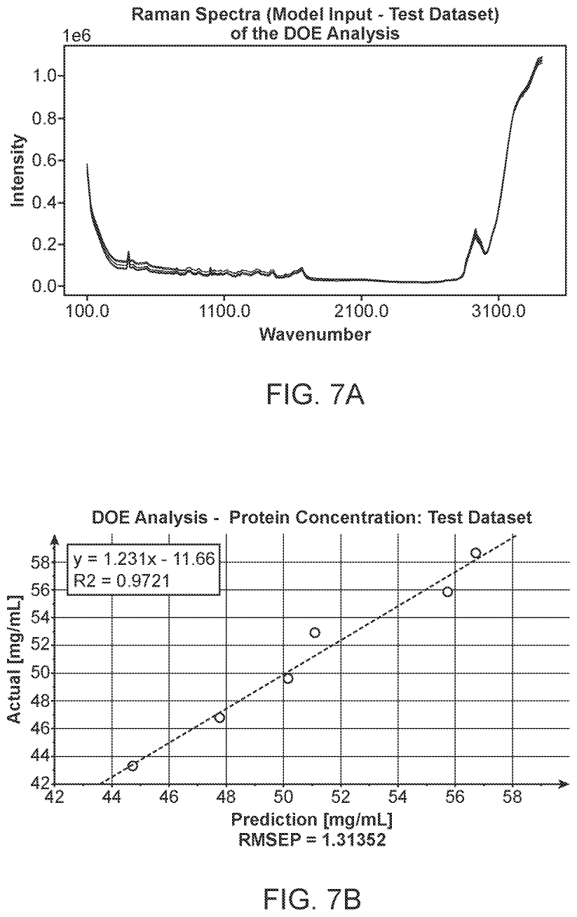

FIG. 7A shows the inline Raman scans or measurements of conditioned UF/DF pool samples 8-13. The protein concentrations for these samples were measured using a protein concentration sensor (SoloVPE). It can be seen that the scans are very consistent and the main variation is attributed to protein concentration changes. The DOE training dataset includes the Raman measurements as well as the protein concentration measurements corresponding thereto. The DOE training dataset was used to build a DOE PLS model using 1st derivative, standard normal variate (SNV) and UV scaling for the wavenumbers 500-1850. As used herein, a UV scaling refers to any modeling process of projecting a surface of 3D mesh to a 2D space for. The DOE PLS model used one latent variable. The DOE PLS model was then tested on the DOE testing dataset of inline Raman measurements of samples 8-13, and the performance comparing the measured protein concentrations to the protein concentrations predicted by the DOE PLS model is shown in FIG. 7B. The correlation coefficient of R2=0.9721 and the small RMSEP=1.31352 confirm that the DOE PLS model has an acceptable performance for protein concentration.

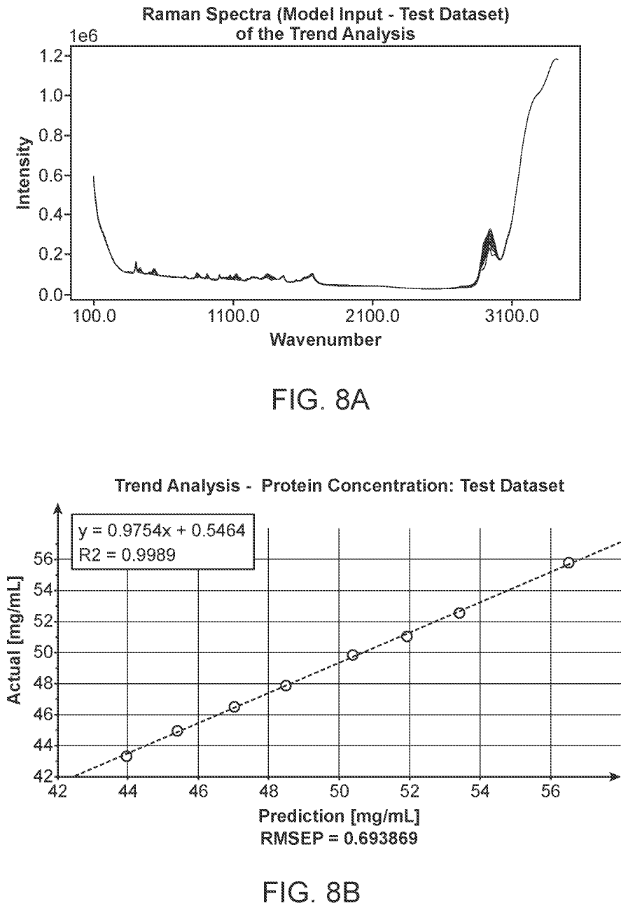

FIG. 8A shows the inline Raman scans or measurements of conditioned UF/DF pool samples 14-21. FIG. 8B shows the performance of a TA PLS model in predicting protein concentrations for a TA testing dataset. A TA PLS model trained using the Raman and protein concentration measurements of UF/DF conditioned pools of samples 22-29 was tested, i.e., used to predict the protein concentrations of UF/DF conditioned pool samples 14-21. The TA PLS model used wavenumbers 500-1850, 1st derivative, SNV and UV scaling for data pretreatment. One latent variable was selected. The R2=0.9989 and RMSEP=0.6938 as shown in FIG. 8B confirm acceptable performance and the feasibility of model scale up (e.g., from 25 mL to 750 mL).

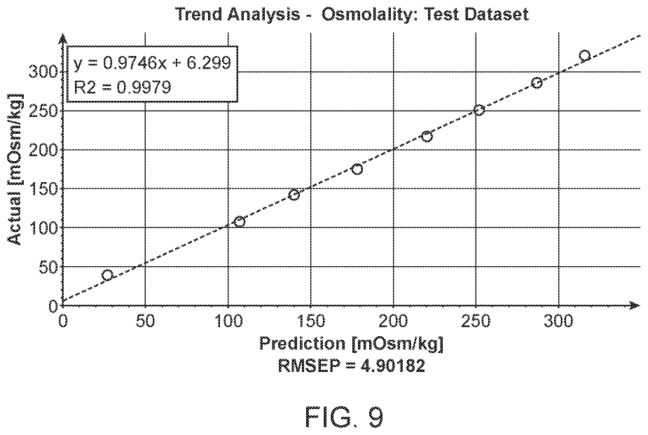

FIG. 9 shows the performance of a TA PLS model in predicting osmolality for a TA testing dataset. A TA PLS model trained using the Raman and osmolality measurements of UF/DF conditioned pools of samples 22-29 was tested, i.e., used to predict the osmolality of UF/DF conditioned pool samples 14-21. The TA PLS model used wavenumbers 500-1500, first derivative, SNV and UV scaling for data pretreatment. One latent variable was selected. The R2=0.9979 and RMSEP=4.90182 as shown in FIG. 9 confirm acceptable performance and feasibility of scale-up (e.g., from 25 mL to 750 mL).

The performance of the DOE PLS model in predicting protein concentrations of the “trend” conditioned UF/DF pool samples (samples 14-29) is typically not as good as the performance of the performance of the TA PLS model (e.g., RMSEP=6.924). This can be seen from a comparison of the measured protein concentrations to the predictions of the DOE PLS model (e.g., using the inline Raman measurements of samples 14-21 or 22-29 as TA testing dataset). The DOE PLS model performance in predicting protein concentration of the TA testing dataset is good when the protein concentration is around 50 g/L, i.e., the end-point, which shares an equivalent matrix composition between DOE and trend analysis. The results indicate that TA PLS model obtained from or trained by a TA training dataset is generally more suited for predicting product quality attributes based on TA testing dataset, i.e., testing datasets including Raman measurements of conditioned UF/DF pools obtained for a range of recovery phase matrix conditions (e.g., beginning-to-end recovery phase matrix conditions), compared to DOE PLS models obtained from or trained by a DPE training dataset.

Similar to the performance of DOE PLS model in predicting protein concentrations of the “trend” conditioned UF/DF pool samples, the performance of the TA PLS model in predicting protein concentrations of the DOE conditioned UF/DF pool samples (samples 8-13) is found to not be as good as that of the DOE PLS model. Except protein concentration ˜50 g/L, i.e., the end-point, which shares an equivalent matrix composition between DOE and trend analysis, the errors in the predictions by the TA PLS model can be larger (e.g., RMSEP=4.713), indicating that TA PLS models obtained from or trained by a TA training dataset are generally not as well suited as DOE PLS models for predicting product quality attributes based on DOE testing dataset, i.e., testing datasets including Raman measurements of conditioned UF/DF pools obtained for a fixed target range of matrix conditions.

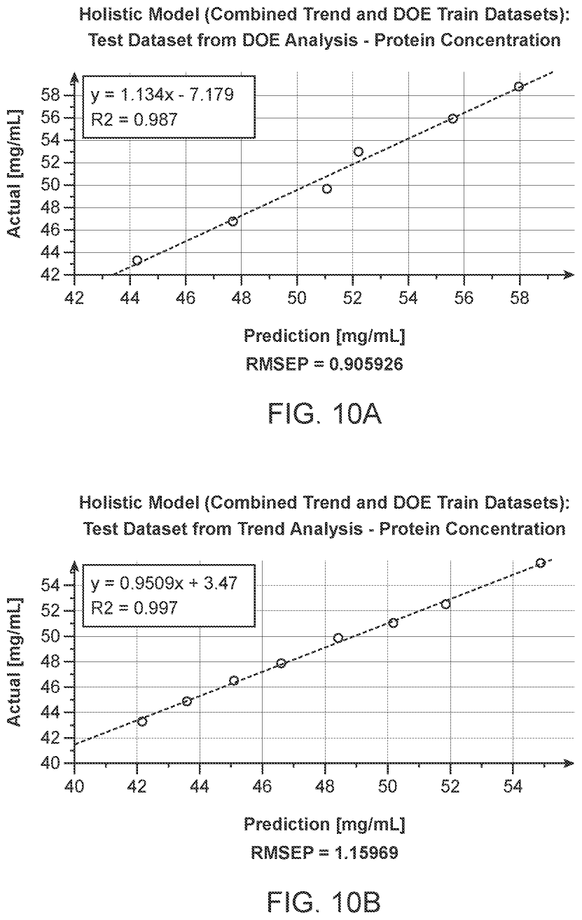

Alternatively, a holistic model including the train data from both DOE and TA can be developed. The holistic model can include both TA and target DOE properties to accurately predict for target and trend conditions. For example, a holistic model including the train data from both DOE (samples 1-7) and TA (samples 22-29) was developed. The holistic model can include both TA and target DOE properties to accurately predict for target and trend conditions simultaneously. A PLS model based on wavenumbers 500-1850 and combination of 1st derivative, SNV and UV Scaling for data pretreatment is developed. Three latent variables are selected using 7-fold cross-validation. The holistic model performance is first evaluated against the DOE test dataset (samples 8-13) in FIG. 10A. The correlation coefficient of R2=0.987 and the small RMSEP=0.905926 confirm that the model has an acceptable performance for protein concentration prediction. Next, the holistic model performance is evaluated against the Process Trend test dataset (samples 14-21) in FIG. 10B. The correlation coefficient of R2=0.997 and the small RMSEP=1.15969 confirm that the model has an acceptable performance for protein concentration prediction.

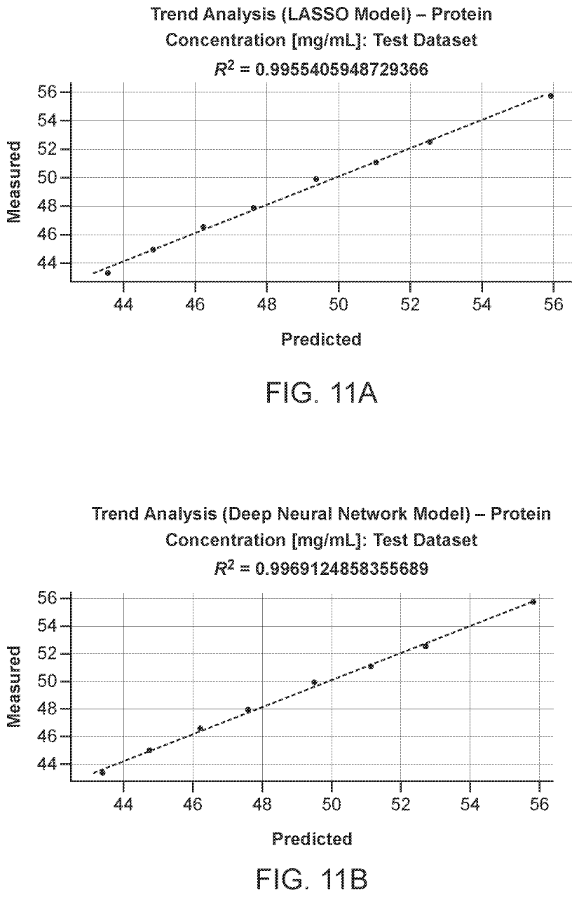

A LASSO model was built using the TA training dataset (conditioned UF/DF pool samples 22-29) with regularization parameter alpha=0.006 as the result of four-fold cross-validation methodology. LASSO regression automatically eliminates the model coefficients that are not contributing to error minimization through a penalty term, alpha. The model was tested against TA test dataset in FIG. 11A (samples 14-21). The R2=0.996 and RMSEP=0.88 confirms that LASSO model can be used as an alternative to the PLS model used in FIG. 6B.

A deep neural network (DNN) model was built using the TA training dataset (conditioned UF/DF pool samples 22-29) to evaluate whether the deployment of a non-linear model that captures non-linear correlations may result in a better performance. The optimal DNN hyper parameters (as the result of hyper-parameter tuning) include an input layer, two hidden layers (each 4 neurons), RELU activation functions for input and hidden layers and linear activation function for the output with L1-0.008 to regularize the input layer to avoid over-fitting. The DNN model predictions of protein concentrations of the Trend testing dataset (conditioned UF/DF pool samples 14-21) in comparison to the measured protein concentrations are depicted in FIG. 11B. The R2=0.997 and RMSEP=0.52 confirms that DNN model can be used as an alternative to the PLS model in FIG. 6B.

The above described data (e.g., training dataset) and resulting models demonstrate that alternative models can be used resulting in similar performance as the above PLS models.

In various embodiments, the capability to predict the product quality attributes of conditioned UF/DF pools in recovery vessels allows one to monitor and control in real-time the recovery operations. An inline spectrometer may perform Raman scans of a conditioned UF/DF pool and transmit the Raman measurements to a computing platform hosting a machine learning model that is trained to predict product quality attributes based on an analysis of the Raman measurements. The computing platform can be on-site or on the premises of the UF/DF recovery vessel or can be communicating with the UF/DF recovery vessel remotely. For example, the computing platform can be an IOT edge node, an on-premise virtual machine, a cloud server, a cloud serverless solution, or combination thereof, capable of communicating with the UF/DF recovery vessel with a latency of less than about 3 seconds, about two seconds, about 1 second, including values and subranges therebetween.

In various embodiments, the machine learning model may predict a product quality attribute (e.g., such as but not limited to protein concentration, osmolality, etc.), of the conditioned UF/DF pools, which may then be signaled to the UF/DF recovery vessel, or control units thereof, with very low latency. For example, the time duration between the inline Raman scans and the arrival of the signals at the recovery vessel, or a control unit thereof, may be less than about 3 seconds, about two seconds, about 1 second, including values and subranges therebetween. As such, the predictions of the product quality attributes can be used for real-time or nearly real-time monitoring of the progress of the conditioned UF/DF recovery operation. Further, the predictions can also be used to control the recovery operations. For example, the computing platform may determine that a different product quality attribute value than the predicted one may indicate an improved quality of the conditioned UF/DF pool, or recovery thereof. In such cases, the computing platform may transmit a signal, for instance a signal configured to cause the addition, or termination thereof, of a buffer into the recovery vessel, to alter or adjust the protein concentration or osmolality of the UF/DF pool to a value that indicates the target or improved quality of the conditioned UF/DF pool.

### V. Example Experimental Demonstration of Real-Time Monitoring and Control of Conditioned UF/DF Pools

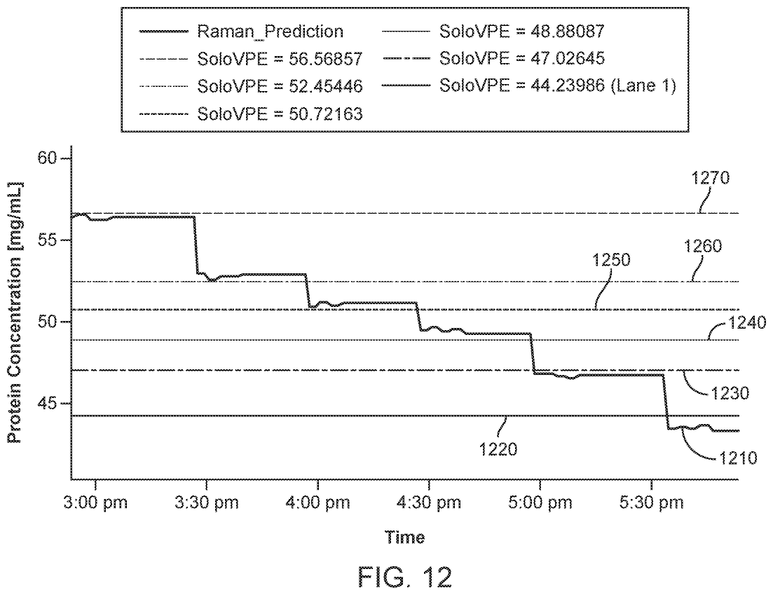

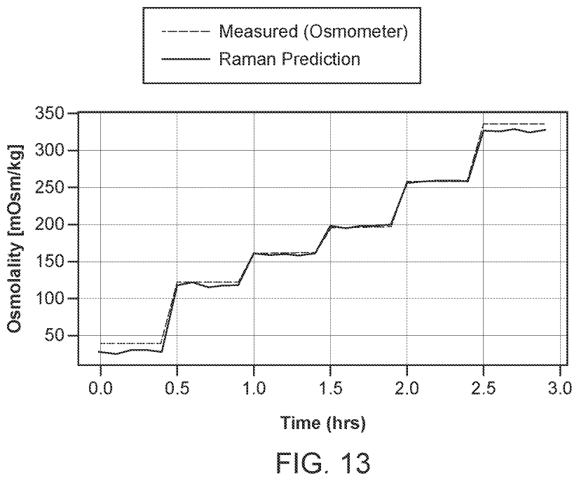

The monitoring of the product quality attributes of a conditioned UF/DF pool is illustrated with reference to FIG. 12 and FIG. 13. FIG. 12 shows time-series plot of experiment data comparing real-time protein concentration predictions using Process Trend model described above versus protein concentration of samples taken throughout the experiment and measured using a protein concentration sensor (e.g., SoloVPE. FIG. 13 shows time-series plot of experiment data comparing osmolality predictions using Raman spectra collected from real-time experiment in FIG. 12 as input to Process Trend model versus osmolality of samples taken throughout the experiment and measured offline using an osmometer. An experiment to demonstrate the disclosed monitoring of a UF/DF pool was conducted using conditions shown in Table 3 below, where the first step corresponds to the filling up of the recovery vessel with the UF/DF diluted pool (mAbs feedstock), and each additional step represents a subsequent dilution with a conditioning buffer (20 mM Sodium Acetate, 1057 mM (40%) Trehalose, 0.20% w/v Polysorbate 20, pH 5.3). The recovery vessel used in the experiment was a vessel typically used for housing and growing cell culture and other media and suitable to store UF/DF pools and receive the conditioning buffers in the amounts specified in the tables below. Furthermore, the vessel was configured to receive an inline or in-situ probe for Raman spectroscopy measurements.

TABLE 3

| | Target Protein | Conditioning Buffer |
|---|---|---|
| Step | Concentration (g/L) | Mass Added (g) |
| 1 | 55.6 | 900.0 |
| 2 | 52.5 | 73.4 |
| 3 | 51 | 37.2 |
| 4 | 49.5 | 35.9 |
| 5 | 46.5 | 66.9 |
| 6 | 43.5 | 88.4 |

Raman scans of the recovery vessel were obtained from the start of the filling of the recovery vessel with conditioning buffer using an inline Raman spectrometer that is operationally coupled to the recovery vessel. The Raman scans were performed at the rate of 1 scan every 3 min, for 15 minutes, i.e., a total of 5 Raman scans per condition. Mixing was conducted throughout the experiment. The Raman measurements were provided to a trained machine learning model that computed the predicted protein concentrations and osmolality of the conditioned UF/DF pool, which are shown respectively in FIG. 12 and in FIG. 13 in comparison to measured values of the product quality attributes. The trained machine learning model was a supervised learning model that was trained using a training dataset comprising a plurality of Raman measurements of a UF/DF pool with known protein concentrations and known osmolality of the UF/DF pool. The supervised machine learning model was trained to receive, as input, a Raman measurement, and produce, as output, a protein concentration and an osmolality of the UF/DF pool. The protein concentrations in FIG. 12 undergo several steps starting from the initial concentration 1270 of 55.6 g/L. At each step of buffer addition, it is shown that the predicted protein concentration 1210 at least substantially matches the protein concentrations 1220-1270 measured by a protein concentration sensor, illustrating that the disclosed methods and systems allow for the real-time monitoring, with the use of inline Raman spectroscopy, of product quality attributes of conditioned UF/DF pools over the course of the recovery operations.

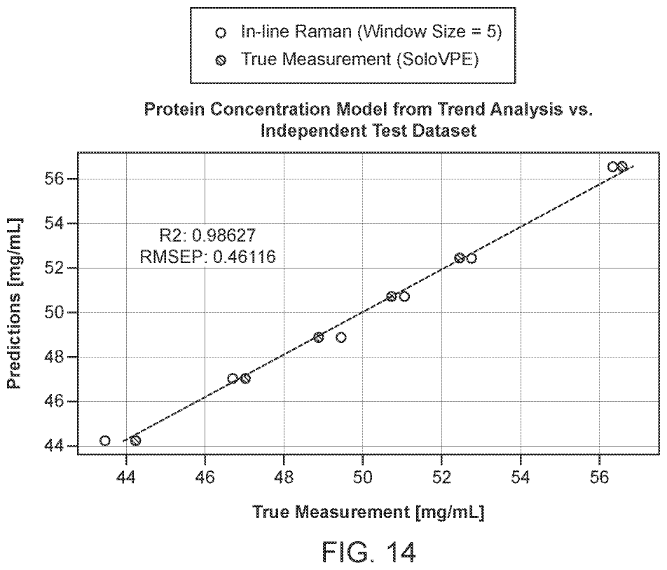

For example, for the first half hour of the UF/DF pool recovery operations, the measured protein concentration 1270 substantially matched the predicted protein concentration 1210 for the same period. Further, once a conditioning buffer was added into the recovery vessel, the protein concentration of the UF/DF pool changed, which was measured with a protein concentration sensor. The protein concentration was also predicted as discussed in the instant application. The measured new protein concentration 1260 was found to substantially match the predicted protein concentration 1210 for that same period. Same applies to further additions of conditioning buffer to the recovery vessel, where the measured protein concentrations 1250, 1240, 1230, and 1220 during the time periods after the additions of the conditioning buffer substantially matched the protein concentrations 1210 predicted for the corresponding time periods. FIG. 14 shows a scatter plot of actual (true measurement) versus predicted (averaged in a window of last five measurements) protein concentration corresponding to the time-series plot in FIG. 12, in accordance with various embodiments. The R2=0.98627 and RMSEP=0.46116 in FIG. 14 confirms the acceptability of the results in FIG. 12.

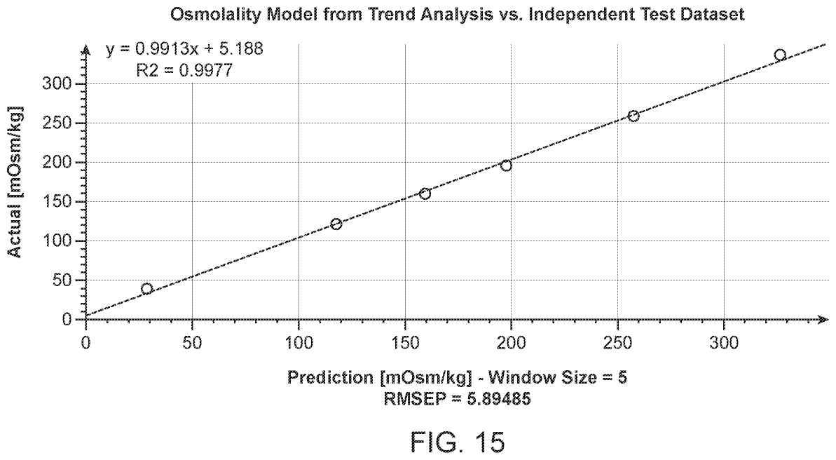

With reference to FIG. 13, an analysis was conducted using the Raman scans collected during the above real-time protein concentration experiment to predict osmolality of the conditioned UF/DF pool. The osmolality of the conditioned UF/DF pool as measured by an osmometer, for samples 1-6 in Table 3, at least substantially matched with the osmolality predicted by the machine learning model, illustrating that the disclosed methods and systems allow for the real-time monitoring of product quality attributes of conditioned UF/DF pools over the course of the recovery operations. FIG. 15 shows a scatter plot of actual versus predicted (averaged in a window of last five measurements) osmolality corresponding to time-series plot in FIG. 13, in accordance with various embodiments. The R2=0.9977 and RMSEP=5.89485 in FIG. 15 confirms the acceptability of the results in FIG. 13.

An experiment to demonstrate the disclosed control of a conditioned UF/DF pool using product quality attribute predictions of a machine learning model was conducted using conditions shown in Table 4 below, where the first step corresponds to the filling up of the recovery vessel with the UF/DF diluted pool (mAbs feedstock), and each additional step represents a subsequent dilution with a conditioning buffer (20 mM Sodium Acetate, 1057 mM (40%) Trehalose, 0.20% w/v Polysorbate 20, pH 5.3). The addition of the protein and buffer into the recovery vessel was fully automated, using a control system at a cloud server in remote communication with the on-site control system of the recovery operation of the conditioned UF/DF pool (using a Kepware® connectivity solution).

TABLE 4

| | Target Protein | Conditioning Buffer |
|---|---|---|
| Step | Concentration (g/L) | Mass Added (g) |
| 1 | 55.6 | 940.0 |
| 2 | 50 | 146.3 |
| 3 | 45 | 148.9 |

At step 1 of the experiment, the recovery vessel was filled with a UF/DF pool having a protein concentration of about 55.6 g/L. The protein concentration of the initial UF/DF pool was determined as discussed above. Initially, the UF/DF pool was scanned with an inline Raman spectrometer, and the Raman measurements were provided to a remote computing platform that was hosting a trained machine learning model and was in communication with the recovery vessel. The Raman scans of the UF/DF pool were then provided to the computing platform so that the trained machine learning model analyzed the Raman measurements and predicted the protein concentration of the UF/DF pool (about 55.6 g/L). The trained machine learning model was a supervised learning model that was previously trained using a training dataset comprising a plurality of Raman measurements of a UF/DF pool with known protein concentrations and known osmolality of the UF/DF pool. The supervised machine learning model was trained to receive, as input a Raman measurement, and produce, as output, a protein concentration and an osmolality of the UF/DF pool.

Step 2 corresponds to the adjustment of the protein concentration of the UF/DF pool in the recovery vessel by the addition of a conditioning buffer. Mixing was conducted throughout the experiment. For example, if a technician operating the recovery operation sought to achieve the target protein concentration of the UF/DF pool at about 50 g/L, the remote computing platform determined the amount of conditioning buffer needed to be added into the recovery vessel to adjust the protein concentration of the UF/DF pool to the desired value (of 50 g/L). The computing platform accessed a mapping (e.g., a formula, a table, a model, etc.) that related weight indicators of the recovery vessel or the UF/DF pool to protein concentration values.

The weight, as indicated by the weight indicator, was generally positively correlated with the protein concentration values. In this example, the weight indicator used was a typical real-time weight sensor used in various bio-processes. This sensor directly provided the weight measurement of the recovery vessel and UF/DF pool as a continuous signal. However, it is contemplated that, in some embodiments, weight can also be indirectly indicated (inferred) from other sensors. For example, the real-time measurement of a vessel height when multiplied by the constant area and density of the material, can be used to provide a real-time estimation of the weight. It is also contemplated that, in some embodiments, the weight indicator can be used to stop the process when a target weight value is achieved. The target weight value can be directly related to target protein concentration given the initial protein concentration and simple mass balancing, considering start and target conditions.

Thus, the computing platform used the mapping to determine or compute a weight indicator of the recovery vessel or the UF/DF pool that corresponds to a protein concentration of 50 g/L. Examples of weight indicators include weight of the recovery vessel (including the UF/DF pool contained therein), height or volume of the UF/DF pool, etc. Using the weight of the recovery vessel as a non-limiting illustrative example, the computing platform determined or computed the weight of the recovery vessel that corresponds to the protein concentration of 50 g/L. The computing platform then determined the amount of buffer that needed to be added into the recovery vessel so that its weight at least substantially matched the determined or computed weight. It is to be appreciated that the computing platform can similarly compute the weight of the recovery vessel for other protein concentrations and determine the amount of buffer that needs to be added into the recovery vessel so that its weight increases to the computed weight. For example, the computing platform can compute the weight of the recovery vessel when the protein concentration is about 55.6 g/L, and determine the amount of buffer that needs to be added into the recovery vessel so that its weight increases to the computed weight (e.g., which corresponds to protein concentration of 50 g/L). After reaching the desired weight, the buffer addition was automatically stopped by stopping the feed pump where a signal was automatically sent from the computing platform to the feed pump to set the flow set point to zero.

Step 3 corresponds to the adjustment of the protein concentration of the UF/DF pool in the recovery vessel from 50 g/L to a second target concentration of 45 g/L using the same process described above with reference to steps 1 and 2.

In some instances, Raman measurements of conditioned UF/DF pools may be taken to evaluate the effectiveness of the buffer addition. For example, these Raman measurements may be processed or analyzed by the machine learning model to predict the protein concentration of the conditioned UF/DF pool, which can then be compared with the target protein concentration to evaluate the effectiveness of the buffer addition. As is to be appreciated, the predicted quality attributes at the target condition was used to decide on the release of the drug substance and allow instant release of the product. For example, the predicted product quality attribute may be compared to a predetermined threshold for the predicted product quality attribute to determine whether the conditioned UF/DF pool is ready for instant release.

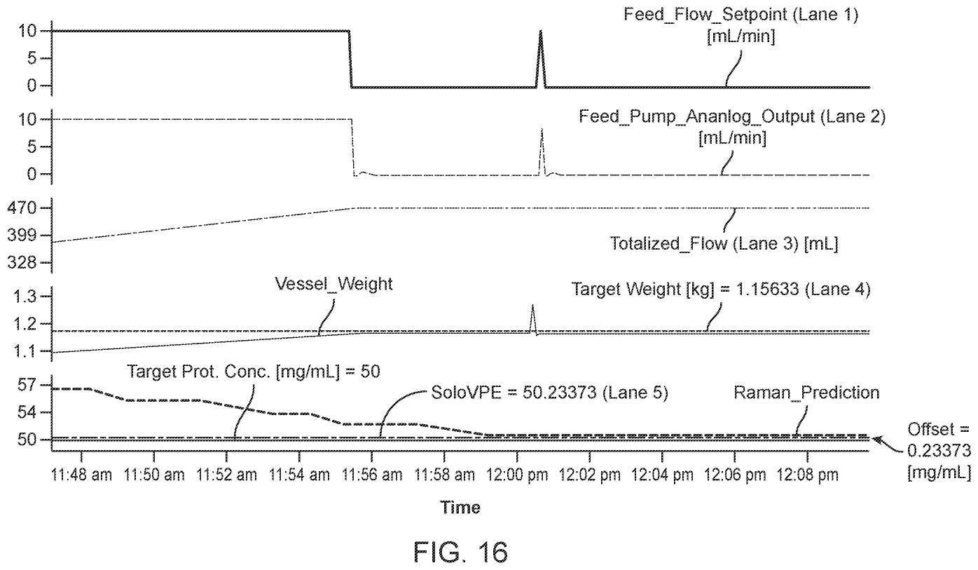

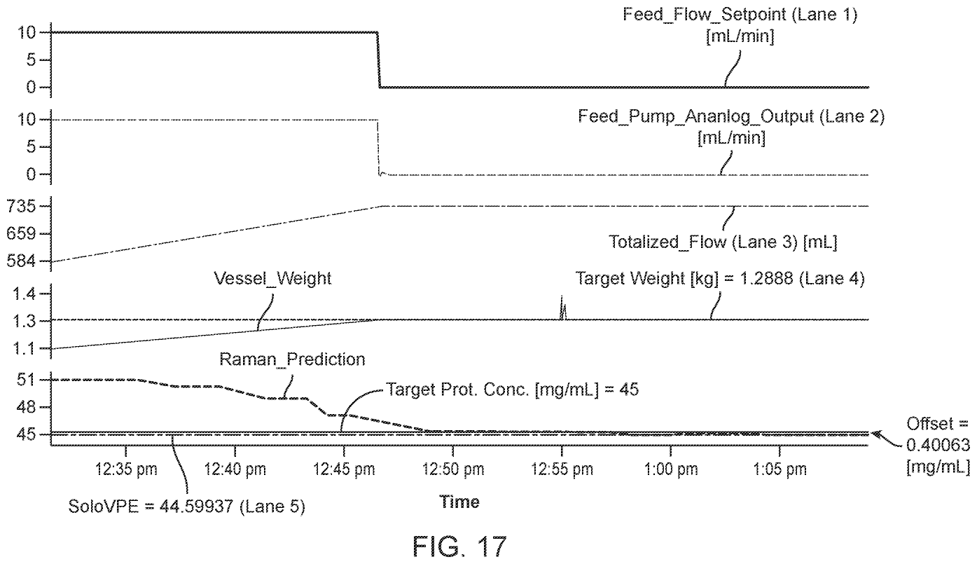

FIGS. 16-17 show plots illustrating real-time control of a conditioned UF/DF pool in a recovery vessel based on real-time regulation of the weight of the recovery vessel. Moreover, as illustrative examples, FIGS. 16 and 17 show results of an experimental demonstration where protein concentrations predicted using the disclosed techniques substantially matched measured protein concentrations at the end of a recovery operation to within 0.5% (an offset of about 0.23373 g/L) in the former case (i.e., the experimental run of FIG. 16) and to within 0.9% (an offset of about 0.40063 g/L) in the latter case (i.e., the experimental run of FIG. 17). As previously discussed, upon determining the amount of buffer to be added into the recovery vessel, the computing platform generates and sends a signal to a buffer pump to cause the buffer pump add buffer in the determined amount and automatically stop when reaching the determined amount. Thus, with respect to steps 1 and 2 of Table 4, the computing platform generated and sent a buffer pump signal to a buffer pump that caused the buffer pump to add about 146.3 g of conditioning buffer and automatically stop after reaching the target weight. Raman measurements of the UF/DF pool after the addition of the buffer were taken in real-time, and the trained machine learning model was used to analyze the Raman measurements and compute the protein concentration. FIG. 16 shows that the addition of the buffer resulted in the conditioned UF/DF pool attaining the target protein concentration of about 50 g/L (with an offset of about 0.23373 g/L). In addition, FIG. 16 shows signals to add buffer, and then automatically stop after reaching the target weight, as indicated by the high and low modes, respectively, of each of the lines corresponding to the Feed_Flow_Setpoint (Lane 1), and Feed_Pump_Analog_Output (Lane 2). Furthermore, FIG. 16 shows the addition of the buffer and the corresponding increase in vessel weight until the vessel weight reaches target weight, as reflected by the lines corresponding to Totalized_Flow (Lane 3) and Vessel_Weight reaching Target Weight (Lane 4).

Similarly, FIG. 17 is another run showing that the addition of the buffer from step 2 to step 3 of table 4 resulted in the conditioned UF/DF pool attaining the target protein concentration of about 45 g/L (with an offset of about 0.40063 g/L). Like FIG. 16, FIG. 17 also shows the signals to add buffer, and then automatically stop the addition after reaching the target weight, as indicated by the high and low modes, respectively, of each of the lines corresponding to the Feed_Flow_Setpoint (Lane 1), and Feed_Pump_Analog_Output (Lane 2). Furthermore, FIG. 17 shows the addition of the buffer and the corresponding increase in vessel weight until the vessel weight reaches a target weight, as reflected by the lines corresponding to Totalized_Flow (Lane 3) and Vessel_Weight reaching Target Weight (Lane 4). FIGS. 16 and 17 illustrate the accuracy of the disclosed techniques for predicting target product quality attributes, where in the former case, the final protein concentration is predicted to within an accuracy of about 99.5%, and in the latter case, the final protein concentration is predicted within an accuracy of about 99.1%. Further, it is to be noted that the entire process from the Raman scanning of the UF/DF pool at step 1 to the determination of the protein concentration (about 45 g/L) of the conditioned UF/DF pool by the machine learning model after the addition of the buffer at step 3 occurred within about 15 minutes. Further, it was also observed that the latency between the Raman measurements being provided to the machine learning model and the buffer pump signals being received at the buffer pump was about 1 second or less. As such, FIGS. 16-17 demonstrate a real-time control of the recovery operation of a UF/DF pool as it is conditioned with a buffer that resulted in its protein concentration going from about 55.6 g/L to about 50 g/L to about 45 g/L (the spikes in FIGS. 16-17 was due to an experimental disturbance). It is to be understood that although the discussion above relates to the control of the recovery operation by predicting protein concentrations of UF/DF pools, the disclosed techniques equally apply when product quality attributes such as osmolality of the UF/DF pools are used to characterize the UF/DF pools.

### VI. Computer Implemented System

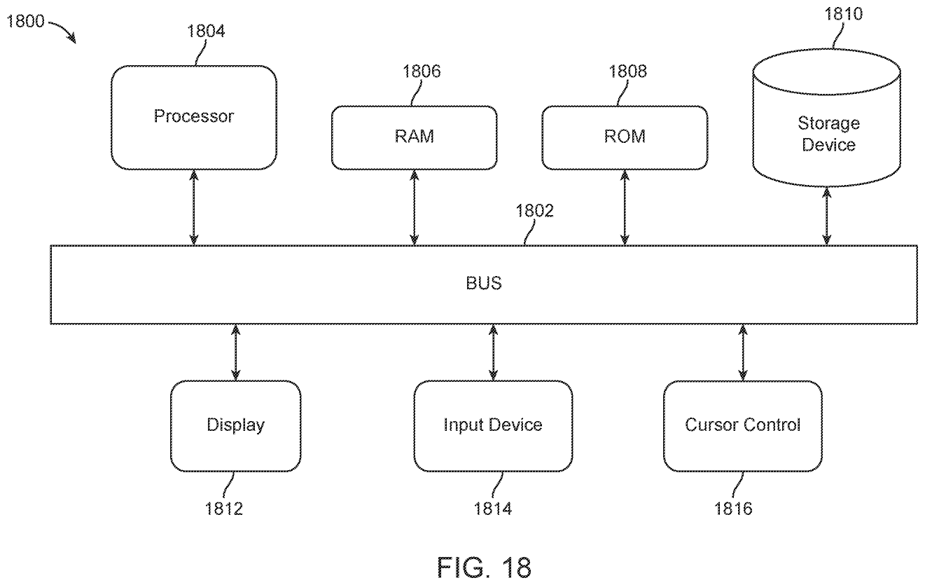

FIG. 18 is a block diagram of a computer system in accordance with various embodiments. Computer system 1800 may be an example of one implementation for product quality attribute prediction system 200 described above in FIG. 2. In one or more examples, computer system 1800 can include a bus 1802 or other communication mechanism for communicating information, and a processor 1804 coupled with bus 1802 for processing information. In various embodiments, computer system 1800 can also include a memory, which can be a random-access memory (RAM) 1806 or other dynamic storage device, coupled to bus 1802 for determining instructions to be executed by processor 1804. Memory also can be used for storing temporary variables or other intermediate information during execution of instructions to be executed by processor 1804. In various embodiments, computer system 1800 can further include a read only memory (ROM) 1808 or other static storage device coupled to bus 1802 for storing static information and instructions for processor 1804. A storage device 1810, such as a magnetic disk or optical disk, can be provided and coupled to bus 1802 for storing information and instructions.

In various embodiments, computer system 1800 can be coupled via bus 1802 to a display 1812, such as a cathode ray tube (CRT) or liquid crystal display (LCD), for displaying information to a computer user. An input device 1814, including alphanumeric and other keys, can be coupled to bus 1802 for communicating information and command selections to processor 1804. Another type of user input device is a cursor control 1816, such as a mouse, a joystick, a trackball, a gesture input device, a gaze-based input device, or cursor direction keys for communicating direction information and command selections to processor 1804 and for controlling cursor movement on display 1812. This input device 1814 typically has two degrees of freedom in two axes, a first axis (e.g., x) and a second axis (e.g., y), that allows the device to specify positions in a plane. However, it should be understood that input devices 1814 allowing for three-dimensional (e.g., x, y and z) cursor movement are also contemplated herein.

Consistent with certain implementations of the present teachings, results can be provided by computer system 1800 in response to processor 1804 executing one or more sequences of one or more instructions contained in RAM 1806. Such instructions can be read into RAM 1806 from another computer-readable medium or computer-readable storage medium, such as storage device 1810. Execution of the sequences of instructions contained in RAM 1806 can cause processor 1804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1804 for execution. Such a medium can take many forms, including but not limited to, non-volatile media, volatile media, and transmission media. Examples of non-volatile media can include, but are not limited to, optical, solid state, magnetic disks, such as storage device 1810. Examples of volatile media can include, but are not limited to, dynamic memory, such as RAM 1806. Examples of transmission media can include, but are not limited to, coaxial cables, copper wire, and fiber optics, including the wires that comprise bus 1802.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1804 of computer system 1800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 1800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1800, whereby processor 1804 would execute the analyses and determinations provided by these engines, subject to instructions provided by any one of, or a combination of, the memory components RAM 1806, ROM, 1808, or storage device 1810 and user input provided via input device 1814.

### VII. Example Implementation of Machine Learning Models

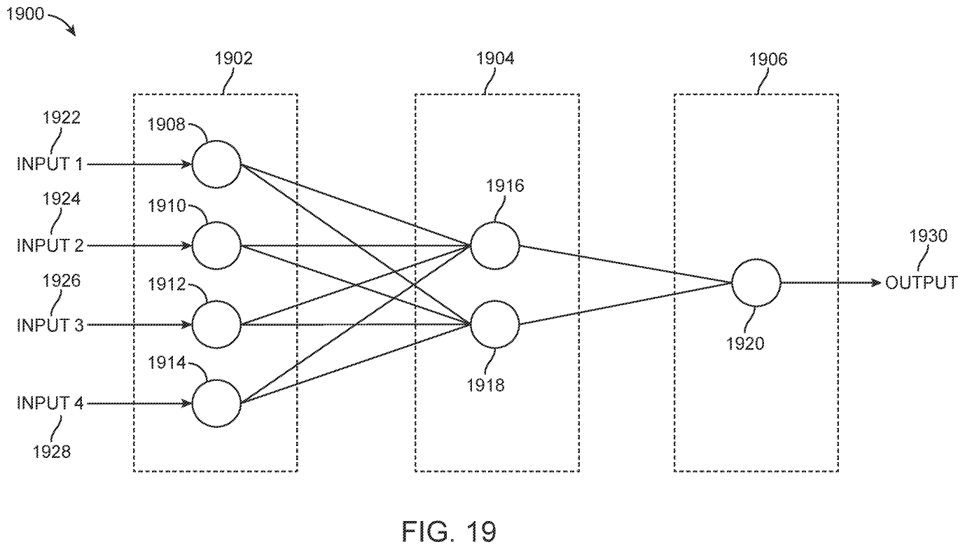

FIG. 19 illustrates an example neural network that can be used to implement a machine learning model according to various embodiments of the present disclosure. For example, in various embodiments, the machine learning model 210 can be implemented using the neural network 1900. As shown, the artificial neural network 1900 includes three layers—an input layer 1902, a hidden layer 1904, and an output layer 1906. Each of the layers 1902, 1904, and 1906 may include one or more nodes. For example, the input layer 1902 includes nodes 1908-1914, the hidden layer 1904 includes nodes 1916-1918, and the output layer 1906 includes a node 1922. In this example, each node in a layer is connected to every node in an adjacent layer. For example, the node 1908 in the input layer 1902 is connected to both of the nodes 1916, 1918 in the hidden layer 1904. Similarly, the node 1916 in the hidden layer is connected to all of the nodes 1908-1914 in the input layer 1902 and the node 1922 in the output layer 1906. Although only one hidden layer is shown for the artificial neural network 1900, it has been contemplated that the artificial neural network 1900 used to implement the machine learning model 210 may include as many hidden layers as necessary or desired.

In this example, the artificial neural network 1900 receives a set of input values 1922-1928 and produces an output value 1930. Each node in the input layer 1902 may correspond to a distinct input value. For example, nodes 1908-1914 in the input layer 1902 may correspond to input values 1922-1928, respectively. In some instances, the input values 1922-1928 may correspond to parameters or values that are provided to the machine learning model 210 as input. In some embodiments, each of the nodes 1916-1918 in the hidden layer 1904 generates a representation, which may include a mathematical computation (or algorithm) that produces a value based on the input values received from the nodes 1908-1914. The mathematical computation may include assigning different weights to each of the data values received from the nodes 1908-1914. The nodes 1916 and 1918 may include different algorithms and/or different weights assigned to the data variables from the nodes 1908-1914 such that each of the nodes 1916-1918 may produce a different value based on the same input values received from the nodes 1908-1914. In some embodiments, the weights that are initially assigned to the features (or input values) for each of the nodes 1916-1918 may be randomly generated (e.g., using a computer randomizer). The values generated by the nodes 1916 and 1918 may be used by the node 1922 in the output layer 1906 to produce an output value for the artificial neural network 1900. When the artificial neural network 1900 is used to implement the machine learning model 210, the output value 1930 produced by the artificial neural network 1900 may correspond to the product quality attribute 212 prediction.

The artificial neural network 1900 may be trained by using training data. For example, the training data herein may be the model training dataset 208. By providing training data to the artificial neural network 1900, the nodes 1916-1918 in the hidden layer 1904 may be trained (adjusted) such that an optimal output is produced in the output layer 1906 based on the training data. By continuously providing different sets of training data, and penalizing the artificial neural network 1900 when the output of the artificial neural network 1900 is incorrect (e.g., when the difference between the predicted product quality attributes 212 and the measured product quality attributes exceeds some threshold), the artificial neural network 1900 (and specifically, the representations of the nodes in the hidden layer 1904) may be trained (adjusted) to improve its performance. In some instances, adjusting the artificial neural network 1900 may include adjusting the weights associated with each node in the hidden layer 1904.

Although the above discussions pertain to an artificial neural network as an example of machine learning, it is understood that other types of machine learning methods may also be suitable to implement the various aspects of the present disclosure. For example, a decision tree, a random forest, a support vector machine, a Bayesian network, a regression model, a multivariate linear model, an ensemble model, etc., or combination thereof, can be used to implement machine learning. The regression model can be a linear regression model (e.g., including multivariate linear regression model), a logistic regression model, a polynomial regression model, a ridge regression model, a least absolute shrinkage and selection operator (LASSO) regression model, a partial least square (PLS) regression model, a principal components regression model, and/or the like. Other types of machine learning algorithms are not discussed in detail herein for reasons of simplicity and it is understood that the present disclosure is not limited to a particular type of machine learning.

### Recitation of Various Embodiments of the Present Disclosure

Embodiment 1: A computer-implemented method of predicting one or more product quality attributes of a conditioned ultrafiltration/diafiltration in a bioprocess, the method comprising: receiving, from a Raman spectrometer operationally coupled to a recovery vessel that contains the conditioned UF/DF pool, a Raman measurement of the conditioned UF/DF pool, wherein the recovery vessel is downstream from an ultrafiltration/diafiltration (UF/DF) operation, and receives a UF/DF pool for conditioning with a buffer to form the conditioned UF/DF pool; and predicting the one or more product quality attributes of the conditioned UF/DF pool using a machine learning model that has been trained to receive, as input, a Raman measurement of a conditioned UF/DF pool and generate, as output, one or more predicted product quality attributes of the conditioned UF/DF pool.

Embodiment 2: The method of embodiment 1, wherein the one or more product quality attributes include osmolality of the conditioned UF/DF pool.

Embodiment 3: The method of embodiment 1 or 2, wherein the conditioned UF/DF pool comprises one or more proteins in a solution, wherein the one or more product quality attributes include protein concentration of the conditioned UF/DF pool.

Embodiment 4: The method of any preceding claim, wherein the UF/DF pool is a purified UF/DF pool received at the recovery vessel from an upstream ultrafiltration and diafiltration process.

Embodiment 5: The method of any preceding claim, wherein the conditioned UF/DF pool includes an antibody.

Embodiment 6: The method of claim 5, wherein the antibody is a monoclonal antibody (mAb).

Embodiment 7: The of any preceding claim, wherein: the machine learning model has been trained to predict the one or more product quality attributes using a training dataset that includes the one or more product quality attributes of conditioned UF/DF pools conditioned with differing amounts of the buffer added therein and associated Raman measurements of the conditioned UF/DF pools conditioned with the differing amounts of the buffer.

Embodiment 8: The method of any preceding claim, wherein the trained machine learning model is a trained multivariate linear model, a trained neural network, a trained deep learning model, or a trained ensemble model.

Embodiment 9: The method of any preceding claim, wherein the trained machine learning model is one of a design of experiments (DOE) partial least squares (PLS) regression model, a trend analytics (TA) partial least squares (PLS) regression model, or a combination thereof.

Embodiment 10: The method of any preceding claim, further comprising: generating an indication indicating whether the conditioned UF/DF pool is ready for instant release based on the one or more predicted product quality attributes.

Embodiment 11: The method of any preceding claim, wherein the steps of receiving the Raman measurement and predicting the one or more product quality attributes is repeated one or more times at a predetermined frequency.

Embodiment 12: A computer-implemented method of controlling an ultrafiltration/diafiltration (UF/DF) pool conditioning process, comprising: receiving, by a processor and from a Raman spectrometer operationally coupled to a recovery vessel that contains a conditioned UF/DF pool, a first Raman measurement of the conditioned UF/DF pool, wherein the recovery vessel is downstream from an UF/DF operation, and receives a UF/DF pool for conditioning with a buffer; predicting, by the processor, using a trained machine learning model receiving the first Raman measurement as an input, one or more product quality attributes of the conditioned UF/DF pool, wherein the one or more product quality attributes include a first protein concentration of the conditioned UF/DF pool; computing, by the processor, a weight indicator of the recovery vessel that corresponds to a second protein concentration of the conditioned UF/DF pool that is different from the first protein concentration of the conditioned UF/DF pool; and outputting, by the processor, an indication of whether to add or cease adding the buffer into the recovery vessel based on the computed weight indicator of the recovery vessel.

Embodiment 13: The method of embodiment 12, wherein the first Raman measurement corresponds to a current time point of the conditioned UF/DF pool.

Embodiment 14: The method of embodiment 12 or embodiment 13, wherein: the processor is in wired or wireless communication with the Raman spectrometer; and a latency between the receiving and the outputting is no greater than about 2s.

Embodiment 15: The method of any of embodiments 12 to 14, wherein the processor is an edge node, a processor that executes a virtual machine, a processor of a cloud server, or a processor of a cloud serverless solution.

Embodiment 16. The method of any of embodiments 12 to 15, wherein the one or more product quality attributes of the conditioned UF/UD pool further include an osmolality of the conditioned UF/DF pool, wherein the predicting includes predicting an osmolality of the conditioned UF/DF pool based on the analysis of the first Raman measurement.

Embodiment 17: The method of any of embodiments 12 to 16, further comprising: receiving, at the processor and from the Raman spectrometer, a second Raman measurement of the conditioned UF/DF pool after the transmitting; predicting, using the processor using the trained machine learning model taking as input the second Raman measurement, a third protein concentration of the conditioned UF/DF pool; and comparing, by the processor, the second protein concentration to the third protein concentration to determine effectiveness of the addition of the buffer into the recovery vessel.

Embodiment 18: The method of any of embodiments 12 to 17, wherein the method does not use any measurement obtained by extracting a sample of the conditioned UF/DF pool from the recovery vessel.

Embodiment 19: The method of any of embodiments 12 to 18, wherein the conditioned UF/DF pool includes an antibody.

Embodiment 20: The method of embodiment 19, wherein the antibody comprises a monoclonal antibody (mAb).

Embodiment 21: The method of any of embodiments 12 to 20, further comprising: responsive to an indication to add the buffer, adding the buffer in an amount sufficient to increase a weight indicator of the recovery vessel to within a weight threshold of the computed weight indicator.

Embodiment 22: The method of any of embodiments 12 to 21, wherein the trained machine learning model is a trained multivariate linear model, a trained neural network, a trained deep learning model, or a trained ensemble model.

Embodiment 23: The method of any of embodiments 12 to 22, wherein the trained machine learning model is one of a design of experiments (DOE) partial least squares (PLS) regression model, a trend analytics (TA) partial least squares (PLS) regression model, or a combination thereof.

Embodiment 24: The method of any of embodiments 12 to 23, further comprising: receiving, at the processor and from the Raman spectrometer, a second Raman measurement of the conditioned UF/DF pool after the outputting; predicting, by the processor using the trained machine learning model taking as input the second Raman measurement, a third protein concentration of the conditioned UF/DF pool; and generating, by the processor, an indication indicating whether the conditioned UF/DF pool is ready for instant release based on the predicted third protein concentration.

Embodiment 25: The method of any of embodiments 12 to 24, further comprising: computing, by the processor, a weight indicator of the recovery vessel that corresponds to the first protein concentration of the conditioned UF/DF; and determining, by the processor, an amount of buffer to add into the recovery vessel based on the weight indicator of the recovery vessel that corresponds to the first protein concentration and the weight indicator of the recovery vessel that corresponds to the second protein concentration.

Embodiment 26: A system, comprising: a ultrafiltration/diafiltration (UF/DF) pool recovery system including: a recovery vessel downstream from an UF/DF operation and configured to receive and store a UF/DF pool for conditioning with a buffer to form the conditioned UF/DF pool; and a Raman spectrometer operationally coupled to the recovery vessel and configured to perform a Raman measurement of the conditioned UF/DF pool; and a communication module operationally coupled to a remote computing platform and the Raman spectrometer and configured to: (i) receive the Raman measurement from the Raman spectrometer and upload the Raman measurement to the remote computing platform for prediction of one or more product quality attributes by the remote computing platform based on the Raman measurement; and (ii) transmit a signal, received from the remote computing platform and related to the one or more product quality attributes, to the UF/DF pool recovery system, wherein: a latency between the receiving of the Raman measurement at the communication module and the transmission of the signal to the UF/DF pool recovery system satisfies a predetermined latency threshold.

Embodiment 27: The system of embodiment 26, further comprising the remote computing platform, wherein the remote computing platform comprises a processor configured to receive the Raman measurement from the communication module, predict one or more product quality attributes, and output a signal related to the one or more product quality attributes.

Embodiment 28: The system of embodiment 27, wherein the one or more product quality attributes include a first protein concentration of the conditioned UF/DF pool; wherein the processor is further configured to compute a weight indicator of the recovery vessel that corresponds to a second protein concentration of the conditioned UF/DF pool that is different from the first protein concentration of the conditioned UF/DF pool; wherein the UF/DF pool recovery system further comprises a buffer pump operationally coupled to the recovery vessel; and wherein the signal is a buffer pump signal configured to instruct the buffer pump to add or cease adding the buffer into the recovery vessel based on a weight indicator of the recovery vessel.

Embodiment 29: The system of embodiment 26, wherein the one or more product quality attributes include an osmolality of the conditioned UF/DF pool.

Embodiment 30: The system of any of embodiments 26 to 29, wherein the latency threshold is about 2 second.

Embodiment 31: The system of any of embodiments 26 to 30, wherein the conditioned UF/DF pool includes an antibody.

Embodiment 32: The system of embodiment 31, wherein the antibody comprises a monoclonal antibody (mAb).

Embodiment 33: A computer-implemented method for providing a tool for predicting a product quality attribute and/or controlling an ultrafiltration/diafiltration (UF/DF) pool conditioning process, the method comprising: obtaining, a training dataset comprising: a plurality of Raman measurements of a conditioned UF/DF pool obtained from a Raman spectrometer operationally coupled to a recovery vessel comprising the conditioned UF/DF pool, wherein the recovery vessel is downstream from an ultrafiltration/diafiltration (UF/DF) operation and receives the UF/DF pool for conditioning with a buffer; and corresponding measurements of one or more product quality attributes of the conditioned UF/DF pool; and training a machine learning model, using said training dataset, to predict the one or more product quality attributes of a conditioned UF/DF pool using as input a Raman measurement of the conditioned UF/DF pool.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

What is claimed is:

1. A computer-implemented method of predicting one or more product quality attributes of a conditioned ultrafiltration/diafiltration (UF/DF) pool in a bioprocess, the method comprising:
receiving, from a Raman spectrometer operationally coupled to a recovery vessel that contains a conditioned UF/DF pool, a Raman measurement of the conditioned UF/DF pool, wherein the recovery vessel is downstream from an ultrafiltration/diafiltration (UF/DF) operation and receives a UF/DF pool conditioned with a buffer; and
predicting the one or more product quality attributes of the conditioned UF/DF pool using a machine learning model that has been trained to receive, as input, a Raman measurement of a conditioned UF/DF pool and generate, as output, one or more predicted product quality attributes of the conditioned UF/DF pool.

2. The method of claim 1, wherein the one or more product quality attributes comprise one or more of:
an osmolality of the conditioned UF/DF pool; or
a protein concentration of the conditioned UF/DF pool.

3. The method of claim 1, wherein the conditioned UF/DF pool includes a monoclonal antibody (mAb).

4. The method of claim 1, wherein:
the machine learning model has been trained to predict the one or more product quality attributes using a training dataset that includes the one or more product quality attributes of conditioned UF/DF pools conditioned with differing amounts of the buffer added therein and associated Raman measurements of the conditioned UF/DF pools conditioned with the differing amounts of the buffer.

5. The method of claim 1, wherein the trained machine learning model is one of a design of experiments (DOE) partial least squares (PLS) regression model, a trend analytics (TA) partial least squares (PLS) regression model, or a combination thereof.

6. The method of claim 1, further comprising generating an indication indicating whether the conditioned UF/DF pool is ready for instant release based on the one or more predicted product quality attributes.

7. The method of claim 1, wherein the steps of receiving the Raman measurement and predicting the one or more product quality attributes is repeated one or more times at a predetermined frequency.

8. A computer-implemented method of controlling an ultrafiltration/diafiltration (UF/DF) pool conditioning process, comprising:
receiving, by a processor and from a Raman spectrometer operationally coupled to a recovery vessel that contains a conditioned UF/DF pool, a first Raman measurement of the conditioned UF/DF pool, wherein the recovery vessel is downstream from an UF/DF operation and receives a UF/DF pool for conditioning with a buffer to form the conditioned UF/DF pool;
predicting, by the processor, using a trained machine learning model receiving the first Raman measurement as an input, one or more product quality attributes of the conditioned UF/DF pool, wherein the one or more product quality attributes include a first protein concentration of the conditioned UF/DF pool;
computing, by the processor, a weight indicator of the recovery vessel that corresponds to a second protein concentration of the conditioned UF/DF pool that is different from the first protein concentration of the conditioned UF/DF pool; and
outputting, by the processor, an indication of whether to add or cease adding the buffer into the recovery vessel based on the computed weight indicator of the recovery vessel.

9. The method of claim 8, wherein:
the processor is in wired or wireless communication with the Raman spectrometer; and
a latency between the receiving and the outputting is no greater than about 2s.

10. The method of claim 8, wherein the one or more product quality attributes of the conditioned UF/UD pool further include an osmolality of the conditioned UF/DF pool, wherein the predicting includes predicting an osmolality of the conditioned UF/DF pool based on the analysis of the first Raman measurement.

11. The method of claim 8, further comprising:
receiving, at the processor and from the Raman spectrometer, a second Raman measurement of the conditioned UF/DF pool after the transmitting;
predicting, using the processor using the trained machine learning model taking as input the second Raman measurement, a third protein concentration of the conditioned UF/DF pool; and
comparing, by the processor, the second protein concentration to the third protein concentration to determine effectiveness of the addition of the buffer into the recovery vessel.

12. The method of claim 8, wherein the method does not use any measurement obtained by extracting a sample of the conditioned UF/DF pool from the recovery vessel.

13. The method of claim 8, wherein the conditioned UF/DF pool includes a monoclonal antibody (mAb).

14. The method of claim 8, further comprising:
responsive to an indication to add the buffer, adding the buffer in an amount sufficient to increase a weight indicator of the recovery vessel to within a weight threshold of the computed weight indicator.

15. The method of any of claim 8, wherein the trained machine learning model is a trained multivariate linear model, a trained neural network, a trained deep learning model, or a trained ensemble model.

16. The method of claim 8, further comprising:
computing, by the processor, a weight indicator of the recovery vessel that corresponds to the first protein concentration of the conditioned UF/DF; and
determining, by the processor, an amount of buffer to add into the recovery vessel based on the weight indicator of the recovery vessel that corresponds to the first protein concentration and the weight indicator of the recovery vessel that corresponds to the second protein concentration.

17. A system, comprising:
a ultrafiltration/diafiltration (UF/DF) pool recovery system including:
a recovery vessel downstream from an UF/DF operation and configured to receive and store a UF/DF pool conditioned with a buffer; and
a Raman spectrometer operationally coupled to the recovery vessel and configured to perform a Raman measurement of the conditioned UF/DF pool; and
a communication module operationally coupled to a remote computing platform and the Raman spectrometer and configured to: (i) receive the Raman measurement from the Raman spectrometer and upload the Raman measurement to the remote computing platform for prediction of one or more product quality attributes by the remote computing platform based on the Raman measurement; and (ii) transmit a signal, received from the remote computing platform and related to the one or more product quality attributes, to the UF/DF pool recovery system, wherein:
a latency between the receiving of the Raman measurement at the communication module and the transmission of the signal to the UF/DF pool recovery system satisfies a predetermined latency threshold.

18. The system of claim 17, further comprising the remote computing platform, wherein the remote computing platform comprises a processor configured to receive the Raman measurement from the communication module, predict one or more product quality attributes, and output a signal related to the one or more product quality attributes.

19. The system of claim 17, wherein the one or more product quality attributes include a first protein concentration of the conditioned UF/DF pool;
wherein the processor is further configured to compute a weight indicator of the recovery vessel that corresponds to a second protein concentration of the conditioned UF/DF pool that is different from the first protein concentration of the conditioned UF/DF pool;
wherein the UF/DF pool recovery system further comprises a buffer pump operationally coupled to the recovery vessel; and
wherein the signal is a buffer pump signal configured to instruct the buffer pump to add or cease adding the buffer into the recovery vessel based on a weight indicator of the recovery vessel.

20. The system of claim 17, wherein the one or more product quality attributes include an osmolality of the conditioned UF/DF pool.
